(19) United States
(12) Patent Application Publication
(10) Pub. No.: US 2006/0216317 A1
(43) Pub. Date: Sep. 28, 2006

(54) Delivery of polynucleotide agents to the central nervous system

(76) Inventors: Christoph Reinhard, Alameda, CA (US); William H. Frey II, White Bear Lake, MN (US)

Correspondence Address: Chiron Corporation;Intellectual Property - R440, P.O. Box 8097, Emeryville, CA, 94662-8097, US

(21) Appl. No.: 11/323,644

(22) Filed: Dec. 29, 2005

Related U.S. Application Data
(63) Continuation of application No. 10/126,060, filed on Apr. 19, 2002, now abandoned.
(60) Provisional application No. 60/285,319, filed on Apr. 20, 2001.
(60) Provisional application No. 60/288,716, filed on May 4, 2001.

(51) Int. Cl. A61K 48/00

(52) U.S. Cl. 424/400; 514/44

(57) ABSTRACT

The present invention provides a method for delivering polynucleotide agents, particularly oligonucleotides, to the CNS of a mammal by way of a neural pathway originating in the nasal cavity or through a neural pathway originating in an extranasal tissue that is innervated by the trigeminal nerve.

### CROSS REFERENCE TO RELATED APPLICATIONS

This application claims the benefit of U.S. Provisional Application Ser. No. 60/285,319, filed Apr. 20, 2001, and U.S. Provisional Application Ser. No. 60/288,716, filed May 4, 2001, both of which are herein incorporated by reference in their entirety.

### FIELD OF THE INVENTION

The present invention is directed to a method for delivering polynucleotide agents to the central nervous system of a mammal by way of a neural pathway originating in either the olfactory region of the nasal cavity, or in an intranasal or extranasal tissue that is innervated by the trigeminal nerve. The disclosed method obviates the drug delivery obstacle imposed by the mammalian blood-brain barrier.

### BACKGROUND OF THE INVENTION

The mammalian brain is characterized by a capillary endothelial cell lining, referred to as the blood-brain barrier (BBB). This monolayer of tight-junctioned endothelial cells provides an anatomical/physiological blood/tissue barrier that prohibits the entry of the majority of solutes present in the blood into the central nervous system (CNS). The anatomical and blood cerebrospinal fluid (CSF) barriers established by the BBB isolates and protects the extracellular fluid (e.g., cerebrospinal fluid) of the brain and spinal cord and their parenchymal tissues from adverse systemic influences, such as infectious blood-borne agents. The BBB also performs a specialized physiologic function that facilities the entry of select molecular species (solutes) into the CNS by establishing endogenous transport systems within the luminal (e.g., contacting the blood compartment) plasma membrane of brain capillaries. More particularly, the human BBB provides a carrier-mediated transport (CMT) pathway for the transport of small molecular weight nutrients required for the sustenance of the cells and tissues comprising the brain and spinal cord parenchyma; and a receptor-mediated transport (RMT) pathway for the transcytosis of large molecular weight protein ligands, such as neurotrophic agents (e.g., growth factors) to the CNS. It has been estimated that the efficiency of the tight-junction connections enables the human BBB to exclude more than 95% of all pharmaceutical agents (e.g., solutes) from entering the CNS from the circulatory system. (Pardridge (1999) Pharmaceutical Science & Technology Today 2:49-59). Thus, it is well known that an agent characterized by a molecular weight over 500 Da which is not lipid-soluble and which lacks an inherent affinity for an RMT system receptor will be unable to cross the BBB.

The morbidity attributed to diseases of the CNS is a major health problem in the United States. Although the use of polynucleotide agents, such as antisense agents, or plasmids comprising a coding sequence for transient protein expression, have been acknowledged as desirable treatment modalities, their development has been hindered by the need for a drug delivery method that is capable of delivering therapeutically effective doses of polynucleotide agents to the CNS. However, within the last few years, there have been several reports documenting the successful use of polynucleotide agents or more specifically, antisense agents for the inhibition of gene expression in the mammalian CNS. For example, antisense-mediated inhibition has been reported for genes encoding such diverse proteins as neurotransmitter receptors, cytokines, transporters and other proteins. Szklerczyk and Kazzmerck (1989) Antisense Nucleic Acid Drug Dev. 9:105.

Conventional approaches for drug delivery to the CNS include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Furthermore, because each of functional/anatomical region of the brain is separated from other regions by hydrophobic white matter, intrastructural injection (e.g., intracerebral or intracerebroventricular injection) promoters very little distribution of an administered agent (e.g., solute) to other regions of the CNS. Thus, there is a continued need for a better method for delivering agents to the tissues and cells of the CNS.

### SUMMARY OF THE INVENTION

The present invention provides a method for delivering polynucleotide agents to the cells and tissues of the CNS of a mammal comprising the step of introducing a preparation comprising a polynucleotide agent into the tissues and cells of the CNS wherein the polynucleotide agent either inhibits the expression of a targeted polypeptide or directs the expression of a protein or peptide that mediates a biological effect on the mammal.

The delivery method disclosed herein provides a method for delivering or administering naked polynucleotides into cells of the CNS (e.g., brain and/or spinal cord) in vivo, comprising the steps of, providing a composition comprising a polynucleotide agent, and contacting the composition with the olfactory region of the nasal cavity, or in an intranasal or an extranasal tissue that is innervated by the trigeminal nerve, wherefrom the polynucleotide agent is delivered to the CNS. More specifically, the invention provides a method for the delivery of polynucleotides to the CNS of a mammal through or by way of neural pathways associated with the olfactory or trigeminal nerves. Suitable polynucleotide agents for use in the delivery/administration methods disclosed herein are preferably DNA or mRNA sequences that encode either a peptide, a protein, or an antisense oligonucleotide.

The polynucleotide agents administered to these sites can be delivered to the CNS in an amount effective to provide a protective or therapeutic effect. Examples of protective or therapeutic effects include protein or peptide expression as well as inhibition of protein expression. Agents delivered according to the method of the invention circumvent the BBB and are delivered directly to the CNS. Accordingly, it is possible to use the method to administer therapeutically effective doses of polynucleotide agents that poorly cross or are unable to cross the BBB for the treatment of a CNS disease or disorder, including but not limited to a neurodegenerative disorder, a malignancy or a tumor, an affective disorder, or nerve damage resulting from a cerebrovascular disorder, injury, or infection of the CNS.

The delivery method provides for the direct transport of exogenous agents into the CNS. In this manner, a polynucleotide agent may be transported along a neural pathway to the CNS, or by way of a perivascular channel, a prelymphatic channel, or a lymphatic channel associated with the brain and/or spinal cord. Alternatively, a polynucleotide agent can enter the cerebrospinal fluid and then subsequently enter the CNS, including the brain, and/or spinal cord.

It is well known that antisense agents provide a means for sequence-specific inhibition of a single gene product. Antisense agents exemplify a particular class of polynucleotide agents that can be delivered according to the method of the invention. As used herein “antisense agents” are sequence-specific regulators designed to inhibit the expression and/or function of a target protein that is known to contribute to the pathology of a CNS disease or disorder. The method can deliver the agent to one or more portions of the CNS as defined herein. Typically, the agent is administered for the prevention or treatment of a CNS disorder or disease.

More specifically, the present invention relates to introduction of naked DNA and RNA (e.g., polynucleotide agents) into a mammal to achieve either the controlled expression of a polypeptide or the in vivo production of an antisense polynucleotide sequence. The delivery method of the invention is useful in gene therapy applications and any therapeutic situation in which either the administration of a polypeptide, or the inhibition of target protein expression could alleviate and/or correct an underlying disorder or disease.

The practice of one embodiment of the present invention requires obtaining naked polynucleotide operatively coding for a polypeptide for incorporation into vertebrate cells. A polynucleotide operatively codes for a polypeptide when it has all the genetic information necessary for expression by a target cell, such as promoters and the like. As used herein these sequences are referred to as plasmids. Suitable polynucleotides for use in the method of the invention can comprise a complete gene, a fragment of a gene, or a composition comprising several genes, together with recognition and other sequences necessary for expression.

In preferred embodiments, polynucleotide agents include nucleotide sequences of sufficient length to encode a full-length protein, or a functional fragment or peptide thereof. In addition, suitable polynucleotide agents also include oligonucleotides designed to be fully complementary to either a region of, or an entire coding sequence of, a mRNA molecule encoding a specific target protein. Accordingly, suitable polynucleotide or oligonucleotide agents for use in the delivery/administration method of the invention include nucleotide sequences of 100, 200, 300, 400, 500, 600, 700, 800, 900, 1000, 1100, 1200, 1300, 1400, 1500, 1600, 1700, 1800, 1900 or 2000 nucleotides in length.

In an alternative embodiment, the invention provides a method for the delivery of antisense agents, for example, a polynucleotide, a chemically modified polynucleotide analogue, or a polynucleotide mimic, to the CNS through an olfactory pathway originating in the olfactory region of the nasal cavity. In a particular embodiment, the method is useful for the administration of compositions comprising one or more antisense agents designed to inhibit the translation of mRNA molecules that encode a target protein whose biological activities contribute to the pathology of a CNS disease or disorder.

More specifically, suitable agents for use in this aspect of the invention include but are not limited to, a polynucleotide (e.g., a single-stranded oligonucleotide), a polynucleotide analogue (e.g., a chemically modified oligonucleotide), or a polynucleotide mimic (e.g., a peptide nucleic acid (PNA) molecule). Each of these species of antisense agent can be utilized either alone or in combination with at least one other antisense agent. Generally speaking, antisense agents are characterized by sequence specificity for a unique portion of the target nucleic acid sequence. Alternatively, a suitable antisense composition may comprise a single type of antisense agent. For example, a composition comprising a single species of polynucleotide, oligonucleotide, or PNA molecule also exemplifies a suitable composition. In addition, a composition comprising two or more polynucleotides, oligonucleotides, or PNA molecules further exemplify a suitable composition for use with the delivery method of the invention.

### DETAILED DESCRIPTION OF THE INVENTION

The delivery method of the present invention preferentially provides for transport of a polynucleotide agent by way of a neural pathway rather than through the circulatory system. By circumventing the BBB, the method of the invention obviates the drug delivery problems imposed by the mammalian BBB and facilitates the direct delivery of agents that are either poorly transported across, or are unable to cross, the BBB. The direct delivery of polynucleotide agents to the CNS using the method of the invention increases the efficiency of delivery and simultaneously decreases the total quantity of agent required for administration. Thus, the disclosed method provides for the direct delivery of therapeutically effective doses of polynucleotide agents and simultaneously minimizes the possibility of unwanted side effects associated with systemic delivery.

More specifically, the invention provides a method for delivering (e.g., transporting) a polynucleotide agent to the CNS of a mammal through or by way of neural pathways associated with the olfactory or trigeminal nerve. The olfactory region is located within the upper one-third of the nasal cavity. An alternative embodiment of the invention involves administering a polynucleotide agent to a tissue that is innervated by the trigeminal nerve.

Transport through or by way of a neural pathway includes intracellular axonal transport and extracellular transport through intercellular clefts in the olfactory neuroepithelium, as well as transport that occurs through or by way of fluid-phase endocytosis by a neuron, through or by way of a lymphatic channel running with a neuron, through or by way of a perivascular space of a blood vessel running with a neuron or neural pathway, through or by way of a mucosal or epithelial cell layer, through or by way of an adventitia of a blood vessel running with a neuron or neural pathway, and transport through the hemangiolymphatic system.

One class of polynucleotide agents useful for the methods of the invention comprises DNA and RNA sequences coding for polypeptides (e.g., peptides and proteins) that have useful therapeutic applications. As used herein the term “naked” polynucleotide agent means that the polynucleotide agents that encode a peptide or protein or antisense polynucletide of interest are free from any delivery vehicle that can act to facilitate entry into the cell, for example, the polynucleotide sequences are free of viral sequences, particularly any viral particles that may carry genetic information. They are similarly free from, or “naked” with respect to, any material that promotes transfection, such as liposomal formulations, charged lipids, or precipitating agents such as calcium phosphate. The term does not exclude the use of polynucleotide agents that comprise a transit peptide to facilitate entry of the agent into a cell.

In general terms, one embodiment of the invention provides a method for obtaining the transitory expression of a polypeptide in the cells of the CNS, comprising the step of introducing a polynucleotide agent comprising a polynucleotide sequence encoding a peptide or protein, whereby the naked polynucleotide may be produced in the cell for weeks and possibly for as long as 30, 45, or 60 days.

Accordingly, polynucleotide sequences that incorporate sequences that direct expression of the polypeptide (e.g., plasmids) are also contemplated within the scope of this invention. Suitable polynucleotide agents for use in the delivery method(s) of the invention include both DNA and mRNA sequences that may or may not encode a peptide or protein. For example, a polynucleotide sequence comprising a plasmid that directs the in vivo production of an antisense polynucleotide can be used in the delivery method of the invention. The DNA sequences used in this embodiment of the method can be sequences that do not integrate into the genome of the host cell. These may be non-replicating DNA sequences, or specific replicating sequences genetically engineered to lack genome-integrating ability. Alternatively, the nucleotide sequences may comprise a synthetic sequence designed to hybrize to an endogenous mRNA molecule in a complementary fashion.

With the availability of automated nucleic acid synthesis equipment, both DNA and RNA can be synthesized directly when the nucleotide sequence is known or by a combination of PCR cloning-and fermentation. Moreover, when the sequence of the desired polypeptide is known, a suitable coding sequence for the polynucleotide can be inferred. Similarly, when a target protein is identified for regulation, a suitable antisense oligonucleotide sequence can also be designed based on the cDNA sequence.

One advantage of in vivo gene therapy based on the use of mRNA is that the polynucleotide agent does not have to penetrate the nucleus to direct protein synthesis; therefore, it should have no genetic liability. The intranasal delivery of mRNA according to the present invention may produce an effect that will generally last at least about 3, 6, 8, or 12 hours. Longer effects can easily be achieved by repeated administration.

Alternatively, in situations requiring a more prolonged effect, an alternative embodiment of the invention provides introducing a DNA sequence coding for a specific polypeptide into the cells of the CNS. Non-replicating DNA sequences that encode peptides, proteins, or antisense agents of interest can be introduced into cells to provide production of the desired polypeptide for periods of about up to about 60 days or 2 months in the absence of genomic integration. Alternatively, an even more prolonged effect can be achieved by introducing the DNA sequence into the cell by means of a vector plasmid having the DNA sequence inserted therein. Preferably, the plasmid further comprises a replicator. Such plasmids are well known to those skilled in the art, for example, plasmid pBR322, with replicator pMB1, or plasmid pMK16, with replicator ColE1 (Ausubel (1988) Current Protocols in Molecular Biology (John Wiley and Sons, New York).

A large number of disease states can benefit from the administration of therapeutic peptides or proteins. Such proteins include lymphokines, such as interleukin-2, tumor necrosis factor, insulin-like growth factor ( e.g., IGF-1), and the interferons; growth factors, such as nerve growth factor, epidermal growth factor, and human growth hormone; tissue plasminogen activator; factor VIII:C; insulin; calcitonin; thymidine kinase; and the like. Moreover, selective delivery of toxic peptides (such as ricin, diphtheria toxin, or cobra venom factor) to diseased or neoplastic cells can have major therapeutic benefits. Current peptide delivery systems suffer from significant problems, such as the necessity of systemically administering large quantities of the peptide (with resultant undesirable systemic side effects) in order to deliver a therapeutic amount of the peptide into or onto the target tissues and cells.

In an alternative embodiment the polynucleotide agents of the invention include DNA or RNA sequences that are themselves therapeutic. Examples of this class of agents include antisense DNA and RNA; DNA coding for an antisense RNA; or DNA coding for tRNA or rRNA to replace defective or deficient endogenous molecules. The polynucleotides of the invention can also code for therapeutic polypeptides. A polypeptide is understood to be any translation product of a polynucleotide regardless of size, and whether glycosylated or not. Therapeutic polypeptides include as a primary example, those polypeptides that can compensate for defective or deficient species in an animal, or those that act through toxic effects to limit or remove harmful cells from the region of interest.

According to the delivery method of the invention, the polynucleotide agent is introduced into the CNS of a mammal through or by way of an olfactory pathway originating in the olfactory region of a mammal's nasal cavity between the central nasal septum and the lateral wall of each main nasal passage. Preferably the agent is delivered to the upper one third of the nasal cavity or to the olfactory epithelium. Agents delivered through or by way of an olfactory pathway can utilize either an intracellular or an extracelluar route. For example an agent may travel along or within an olfactory nerve, an olfactory neural pathway, an olfactory epithelium pathway, or a blood vessel lymphatic channel (e.g., a channel of the hemangiolymphatic system) to access the CNS. For example, once the agent is dispensed into, or onto, the nasal mucosa (e.g., neuroepithelium), the agent may transport through the nasal mucosa and/or olfactory epithelium and travel along olfactory neurons into the CNS.

An alternative embodiment provides for the delivery of a polynucleotide agent to the CNS through or by way of a trigeminal nerve pathway originating from a tissue innervated by the trigeminal nerve. Suitable tissues include both intranasal tissue located within the nasal cavity and extranasal tissues that are innervated by one of the branches (e.g., opthalmic nerve, maxillary nerve, and mandibular nerve) of the trigeminal nerve. As used herein the term “extranasal tissue” refers to, but is not limited to an oral tissue, a dermal tissue, or a conjunctival tissue.

As discussed below, the trigeminal nerve has three major branches, the ophthalmic nerve, the maxillary nerve, and the mandibular nerve. The method of the invention can administer a polynucleotide agent to an intranasal or extranasal tissue that is innervated by one or more of these branches. For example, the method can administer a polynucleotide agent to skin, epithelium, or mucosa of, or around, the face, the eye, the oral cavity, the nasal cavity, the sinus cavities, or the ear.

One embodiment of the present method includes the administration of an polynucleotide agent to a mammal in a manner such that the agent is transported to the CNS in an amount effective to provide a protective or therapeutic effect on a cell or tissue of the CNS. For example, the method can be used to deliver an antisense molecule designed to inhibit the translation of a mRNA molecule that encodes a protein which is known to contribute to the pathology of a CNS disorder. Accordingly, the method of the present invention can be used for the treatment of neurological disorders and psychiatric conditions such as neurodegenerative disorders, malignancies, tumors, affective disorders, or tissue damage resulting from a cerebrovascular disorder, injury, or infection of the CNS.

Use of a neural pathway to transport a polynucleotide agent to the CNS obviates the obstacle presented by the BBB and allows alternative classes of potentially therapeutic molecules, such as chimeric antisense oligonucleotides, to be delivered to the tissues and cells of the mammalian CNS. Although the agent that is administered may also be absorbed into the bloodstream, the sequence specificity and molecular characteristics of a suitable antisense agent will minimize the likelihood of adverse systemic side effects. In addition, because an agent administered according to the disclosed method is not diluted into the fluid volume of the blood compartment of the circulatory system, the invention provides for the delivery of a higher concentration of the agent to the tissues and cells of the CNS than could be achieved using a systemic method of administration. As such, the invention provides an improved method for delivering polynucleotide agents to the CNS.

Neural Pathways

The Olfactory Nerve

The method of the invention includes administration of a polynucleotide agent to tissue innervated by the olfactory nerve. The polynuceotide agent can be delivered to the olfactory area via delivery to the nasal cavity. Preferably, the polynucleotide agent is contacted with the olfactory region of the nasal cavity by instilling the agent to the upper third of the nasal cavity or to the olfactory epithelium. Agents contacted with the olfactory region of a mammal's nasal cavity are delivered to the CNS through or by way of an olfactory nerve pathway, an olfactory epithelium pathway, a perivascular channel, or a lymphatic channel running along the olfactory nerve.

Fibers of the olfactory nerve are unmyelinated axons of olfactory receptor cells that are located in the very top (i.e., superior one-third) of the nasal cavity just under the cribiform plate of the ethmoid bone that separates the nasal and cranial cavities. The olfactory epithelium is the only site in the body where an extension of the CNS comes into direct contact with the external microenviromnent. The dendrites of these sensory neurons extend into the nasal cavity, and the axons collect into nerve bundles that project to the olfactory bulb. The olfactory receptor cells are bipolar neurons with swellings covered by immobile hair-like cilia that project into the nasal cavity. At the other end, axons from these cells collect into aggregates and enter the cranial cavity at the roof of the nose. Surrounded by a thin tube of pia, the olfactory nerves cross the subarachnoid space containing cerebral spinal fluid (CSF) and enter the inferior aspects of the olfactory bulbs. Once the polynucleotide agent is dispensed into/contacted with the nasal cavity, particularly to the upper third of the nasal cavity, the agent can undergo transport through the nasal mucosa and into the olfactory bulb. The olfactory bulb has a widespread connection with various anatomical regions of the brain including but not limited to the anterior olfactory nucleus, frontal cortex, hippocampal formation, amygdaloid nuclei, nucleus of Meynert and the hypothalamus.

The Olfactory Neural Pathway

Thus, in some embodiments the delivery method of the invention includes administration of a polynucleotide agent to a mammal in a manner such that the agent is transported to the CNS along an olfactory pathway (e.g., an olfactory nerve pathway, an olfactory epithelial pathway, or an olfactory region lymphatic channel) originating in the olfactory region of the nasal cavity. Delivery through an olfactory pathway can employ movement of an agent into or across mucosa (e.g., epithelium), through or by way of the olfactory nerve, through or by way of a lymphatic channel, or by way of a perivascular space surrounding a blood vessel that travels with the olfactory nerve to the brain and from there into meningial lymphatics associated with various anatomical regions of the CNS.

Olfactory neurons provide a direct connection to the CNS, brain, and/or spinal cord due, it is believed, to their role in olfaction. CNS delivery through or by way of the olfactory nerve relies on the anatomical connection of the nasal submucosa and the subarachnoid space. A polynucleotide agent administered by the method of the present invention that enters a receptor cell can be transported by way of the fascicles of the olfactory nerve to the rhinoencephalon, which is the portion of the brain that contains the olfactory bulb and structures of the limbic system as well as most of the forebrain. More specifically, administration according to the method of the invention can employ extracellular or intracellular (e.g., transneuronal) axonal transport including anterograde (away-from the cell body and toward the axon terminal) and retrograde (from the axonal terminal to the cell body) transport.

The olfactory mucosa (epithelium) comprises pseudo-stratified columnar epithelium comprised of three principal cell types: receptor cells, supporting cells, and basal cells. Mathison et al. (1998) J. Drug Target 6(6):415. The receptor cell is also referred to as the olfactory cell or primary olfactory neuron. In one embodiment of the method, a polynucleotide agent is administered to the upper third of the nasal cavity in a region located between the central nasal septum and the lateral wall of each main nasal passage. Application of the agent to a tissue innervated by the olfactory nerve can deliver the agent to damaged or diseased neurons or cells of the CNS, brain, and/or spinal cord. For example, an agent contacted with a nasal cavity tissue innervated by the olfactory nerve can be absorbed or transported through the tissue and be delivered to an anatomical region of the CNS such as the brain stem, the cerebellum, the spinal cord, the olfactory bulb, and cortical or subcortical structures.

Agents administered according to the method of the invention can also be delivered to the CNS through or by way of an olfactory mucosal (epithelial) pathway by receptor-mediated transcytosis or by paracellular transport. Alternatively a polynucleotide agent administered according to the method of the invention can be delivered to the CNS through or by way of a supporting cell by pinocytosis or diffusion. In an alternative embodiment, a polynucleotide agent may enter the lamina propia via a paracellular mechanism that permits access to the intercellular fluid. In addition, the perivascular pathway and/or a hemangiolymphatic pathway, such as lymphatic channels running within the adventitia of cerebral blood vessels, provides another possible pathway for the trarnsport of polynucleotide agents to the brain and spinal cord from tissue innervated by the olfactory nerve.

The Trigeminal Nerve

An alternative embodiment of the delivery method of the invention administers a polynucleotide agent to a tissue innervated by the trigeminal nerve. The method of the invention can administer the agent to a tissue that is located within or outside of the nasal cavity and which is innervated by one or more of the branches of the trigeminal nerve. Branches of the trigeminal nerve that innervate tissues outside the nasal cavity include the ophthalmic nerve, the maxillary nerve, and the mandibular nerve. More specifically, in addition to innervating tissues of the nasal cavity (located primarily in the lower two thirds of the cavity), the trigeminal nerve innervates tissues of a mammal's (e.g., a human's) head including skin of the face and scalp, oral tissues, and tissues of and surrounding the eye. Tissues located outside of the nasal cavity that are innervated by the trigeminal nerve include extranasal tissue that is innervated by the trigeminal nerve and extranasal tissue that surrounds the trigeminal nerve. Similarly, epithelium outside the nasal cavity is referred to herein as extranasal epithelium, mucosa outside the nasal cavity is referred to herein as extranasal mucosa, and skin or dermal tissue outside the nasal cavity is referred to herein as extranasal skin or dermal tissue.

The Ophthalmic Nerve and Its Branches

The method of the invention can administer a polynucleotide agent to tissue innervated by the ophthalmic nerve branch of the trigeminal nerve. The ophthalmic nerve innervates tissues including superficial and deep parts of the superior region of the face, such as the eye, the lacrimal gland, the conjunctiva, and skin of the scalp, forehead, upper eyelid, and nose.

The ophthalmic nerve has three branches known as the nasociliary nerve, the frontal nerve, and the lacrimal nerve. The method of the invention can administer the agent to tissue innervated by the one or more of the branches of the ophthalmic nerve. The frontal nerve and its branches innervate tissues including the upper eyelid, the scalp, particularly the front of the scalp, and the forehead, particularly the middle part of the forehead. The nasociliary nerve forms several branches including the long ciliary nerves, the ganglionic branches, the ethmoidal nerves, and the infratrochlear nerve. The long ciliary nerves innervate tissues including the eye. The posterior and anterior ethmoidal nerves innervate tissues including the ethmoidal sinus and the inferior two-thirds of the nasal cavity. The infratrochlear nerve innervates tissues including the upper eyelid and the lacrimal sack. The lacrimal nerve innervates tissues including the lacrimal gland, the conjunctiva, and the upper eyelid. Preferably, the present method administers the agent to the ethmoidal nerve.

The Maxillary Nerve and Its Branches

The method of the invention can administer a polynucleotide agent to tissue innervated by the maxillary nerve branch of the trigeminal nerve. The maxillary nerve innervates tissues including the roots of several teeth and facial skin, such as skin on the nose, the upper lip, the lower eyelid, over the cheekbone, and over the temporal region. The maxillary nerve has branches including the infraorbital nerve, the zygomaticofacial nerve, the zygomaticotemporal nerve, the nasopalatine nerve, the greater palatine nerve, the posterior superior alveolar nerves, the middle superior alveolar nerve, and the interior superior alveolar nerve. The method of the invention can administer the agent to tissue innervated by the one or more of the branches of the maxillary nerve.

The infraorbital nerve innervates tissue including skin on the lateral aspect of the nose, upper lip, and lower eyelid. The zygomaticofacial nerve innervates tissues including skin of the face over the zygomatic bone (cheekbone). The zygomaticotemporal nerve innervates tissue including the skin over the temporal region. The posterior superior alveolar nerves innervate tissues including the maxillary sinus and the roots of the maxillary molar teeth. The middle superior alveolar nerve innervates tissues including the mucosa of the maxillary sinus, the roots of the maxillary premolar teeth, and the mesiobuccal root of the first molar tooth. The anterior superior alveolar nerve innervates tissues including the maxillary sinus, the nasal septum, and the roots of the maxillary central and lateral incisors and canine teeth. The nasopalantine nerve innervates tissues including the nasal septum. The greater palatine nerve innervates tissues including the lateral wall of the nasal cavity. Preferably, the present method administers the agent to the nasopalatine nerve and/or greater palatine nerve.

The Mandibular Nerve and Its Branches

The method of the invention can administer the agent to tissue innervated by the mandibular nerve branch of the trigeminal nerve. The mandibular nerve innervates tissues including the teeth, the gums, the floor of the oral cavity, the tongue, the cheek, the chin, the lower lip, tissues in and around the ear, the muscles of mastication, and skin including the temporal region, the lateral part of the scalp, and most of the lower part of the face.

The mandibular nerve has branches including the buccal nerve, the auriculotemporal nerve, the inferior alveolar nerve, and the lingual nerve. The method of the invention can administer the agent to one or more of the branches of the mandibular nerve. The buccal nerve innervates tissues including the cheek, particularly the skin of the cheek over the buccinator muscle and the mucous membrane lining the cheek, and the mandibular buccal gingiva (gum), in particular the posterior part of the buccal surface of the gingiva. The auriculotemporal nerve innervates tissues including the auricle, the external acoustic meatus, the tympanic membrane (eardrum), and skin in the temporal region, particularly the skin of the temple and the lateral part of the scalp. The inferior alveolar nerve innervates tissues including the mandibular teeth, in particular the incisor teeth, the gingiva adjacent the incisor teeth, the mucosa of the lower lip, the skin of the chin, the skin of the lower lip, and the labial mandibular gingivae. The lingual nerve innervates tissues including the tongue, particularly the anterior two-thirds of the tongue, the floor of the mouth, and the gingivae of the mandibular teeth. Preferably, the method of the invention administers the agent to one or more of the inferior alveolar nerve, the buccal nerve, and/or the lingual nerve.

Tissues Innervated by the Trigeminal Nerve

The method of the invention can administer a polynucleotide agent to any of a variety of tissues innervated by the trigeminal nerve. For example, the method can administer the agent to skin, epithelium, or mucosa of or around the face, the eye, the oral cavity, the nasal cavity, the sinus cavities, or the ear.

Thus, in one embodiment, the method of the invention administers a polynucleotide agent to skin innervated by the trigeminal nerve. For example, the present method can administer the agent to skin of the face, scalp, or temporal region. Suitable skin of the face includes skin of the chin; the upper lip, the lower lip; the forehead, particularly the middle part of the forehead; the nose, including the tip of the nose, the dorsum of the nose, and the lateral aspect of the nose; the cheek, particularly the skin of the cheek over the buccinator muscle or skin over the cheek bone; skin around the eye, particularly the upper eyelid and the lower eyelid; or a combination thereof. Suitable skin of the scalp includes the front of the scalp, scalp over the temporal region, the lateral part of the scalp, or a combination thereof. Suitable skin of the temporal region includes the temple and scalp over the temporal region.

In another embodiment, the method of the invention administers a polynucleotide agent to mucosa or epithelium innervated by the trigeminal nerve. For example, the present method can administer the polynucleotide agent to mucosa or epithelium of or surrounding the eye, such as mucosa or epithelium of the upper eyelid, the lower eyelid, the conjunctiva, the lacrimal system, or a combination thereof. The method of the invention can also administer the polynucleotide agent to mucosa or epithelium of the sinus cavities and/or nasal cavity, such as the inferior two-thirds of the nasal cavity and the nasal septum. The method of the invention can also administer the agent to mucosa or epithelium of the oral cavity, such as mucosa or epithelium of the tongue; particularly the anterior two-thirds of the tongue and under the tongue; the cheek; the lower lip; the upper lip; the floor of the oral cavity; the gingivae (gums), in particular the gingiva adjacent the incisor teeth, the labial mandibular gingivae, and the gingivae of the mandibular teeth; or a combination thereof.

In yet another embodiment, the method of the invention administers the polynucleotide agent to mucosa or epithelium of the nasal cavity. Other preferred regions of mucosa or epithelium for administering the polynucleotide agent include the tongue, particularly sublingual mucosa or epithelium, the conjunctiva, the lacrimal system, particularly the palpebral portion of the lacrimal gland and the nasolacrimal ducts, the mucosa of the lower yield, the mucosa of the cheek, or a combination thereof.

In other embodiments, the method of the invention administers a polynucleotide agent to nasal tissues innervated by the trigeminal nerve. For example, the present method can be used to administer an agent to nasal tissues including the sinuses, the inferior two-thirds of the nasal cavity, and the nasal septum. Preferably, the nasal tissue for administering the agent includes the inferior two-thirds of the nasal cavity and the nasal septum.

The method of the invention encompasses administration of a polynucleotide agent to oral tissues innervated by the trigerninal nerve. For example, the present method can also administer the agent to oral tissues such as the teeth, the gums, the floor of the oral cavity, the cheeks, the lips, the tongue, particularly the anterior two-thirds of the tongue, or a combination thereof. Suitable teeth include mandibular teeth, such as the incisor teeth. Suitable portions of the teeth include the roots of several teeth, such as the roots of the maxillary molar teeth, the maxillary premolar teeth, the maxillary central and lateral incisors, the canine teeth, and the mesiobuccal root of the first molar tooth, or a combination thereof. Suitable portions of the lips include the skin and mucosa of the upper and lower lips. Suitable gums include the gingiva adjacent the incisor teeth, and the gingivae of the mandibular teeth, such as the labial mandibular gingivae, or a combination thereof. Suitable portions of the cheek include the skin of the cheek over the buccinator muscle, the mucous membrane lining the cheek, and the mandibular buccal gingiva (gum), in particular the posterior part of the buccal surface of the gingiva, or a combination thereof. Preferred oral tissue for administering the polynucleotide agent includes the tongue, particularly sublingual mucosa or epithelium, the mucosa inside the lower lip, the mucosa of the cheek, or a combination thereof.

In another embodiment, the method of the invention administers a polynucleotide agent to a tissue of or around the eye that is innervated by the trigeminal nerve. For example, the present method can administer the agent to tissue including the eye, the conjunctiva, the lacrimal gland including the lacrimal sack, the skin or mucosa of the upper or lower eyelid, or a combination thereof. Preferred tissue of or around the eye for administering the agent includes the conjunctiva, the lacrimal system, the skin or mucosa of the eyelid, or a combination thereof. A polynucleotide agent that is administered conjunctivally but not absorbed through the conjunctival mucosa can drain through nasolacrimal ducts into the nose, where it can be transported to the CNS, brain, and/or spinal cord as though it had been intranasally administered.

The method of the invention also encompasses administration of a polynucleotide agent to a tissue of or around the ear that is innervated by the trigeminal nerve. For example, the present method can administer the agent to tissue including the auricle, the external acoustic meatus, the tympanic membrane (eardrum), and the skin in the temporal region, particularly the skin of the temple and the lateral part of the scalp, or a combination thereof. Preferred tissue of or around the ear for administering the polynucleotide agent includes the skin of the temple.

The Trigeminal Neural Pathway

Thus, in some embodiments the delivery method of the invention includes administration of a polynucleotide agent to a mammal in a manner such that the agent is transported into the CNS, including the brain, and/or spinal cord along a trigeminal neural pathway originating in a tissue that can be located either within or outside of the nasal cavity. Typically, such an embodiment includes administering the agent to a tissue located outside the nasal cavity (i.e., extranasal tissue), which is innervated by the trigeminal nerve. The trigeminal neural pathway innervates various tissues of the head and face, as described above. In particular, the trigeminal nerve innervates the nasal, sinusoidal, oral and conjunctival mucosa or epithelium, and the skin of the face. Application of the agent to a tissue innervated by the trigeminal nerve can deliver the agent to damaged or diseased neurons or cells of the CNS, including the brain, and/or spinal cord. Trigeminal neurons innervate these tissues and can provide a direct connection to the CNS, brain, and/or spinal cord due, it is believed, to their role in the common chemical sense including mechanical sensation, thermal sensation, and nociception (for example detection of hot spices and of noxious chemicals).

Delivery through the trigeminal neural pathway can employ lymphatic channels that travel with the trigeminal nerve to the pons, olfactory area and other brain areas and from there into dural lymphatics associated with portions of the CNS, such as the spinal cord. A perivascular pathway and/or a hemangiolymphatic pathway, such as lymphatic channels running within the adventitia of cerebral blood vessels, provides an additional mechanism for the transport of therapeutic agents to the spinal cord from tissue innervated by the trigeminal nerve.

The trigeminal nerve includes large diameter axons, which mediate mechanical sensation, e.g., touch, and small diameter axons, which mediate pain and thermal sensation, both of whose cell bodies are located in the semilunar (or trigeminal) ganglion or the mesencephalic trigeminal nucleus in the midbrain. Certain portions of the trigeminal nerve extend into the nasal cavity, oral, and conjunctival mucosa and/or epithelium. Other portions of the trigeminal nerve extend into the skin of the face, forehead, upper eyelid, lower eyelid, dorsum of the nose, side of the nose, upper lip, cheek, chin, scalp and teeth. Individual fibers of the trigeminal nerve collect into a large bundle, travel underneath the brain and enter the ventral aspect of the pons. Another portion of the trigeminal nerve enters the CNS in the olfactory area of the brain.

A polynucleotide agent can be administered to the trigeminal nerve, for example, through the nasal cavity's, oral, lingual, and/or conjunctival mucosa and/or epithelium; or through the skin of the face, forehead, upper eyelid, lower eyelid, dorsum of the nose, side of the nose, upper lip, cheek, chin, scalp and teeth. Such administration can employ extracellular or intracellular (e.g., transneuronal) anterograde and retrograde transport of the agent entering through the trigeminal nerves to the brain and its meninges, to olfactory area of the brain, the brain stem, or to the spinal cord. Once the agent is dispensed into or onto tissue innervated by the trigeminal nerve, it may transport through the tissue and travel along trigeminal neurons into areas of the CNS.

Delivery through the trigeminal neural pathway can employ movement of an agent across skin, mucosa, or epithelium into the trigeminal nerve or into a lymphatic, a blood vessel perivascular space, a blood vessel adventitia, or a blood vessel lymphatic that travels with the trigeminal nerve to the olfactory area of the brain and/or pons and from there into meningial lymphatics associated with portions of the CNS such as the spinal cord. Blood vessel lymphatics include lymphatic channels that are around the blood vessels on the outside of the blood vessels. As mentioned above, this also is referred to as the hemangiolymphatic system.

Routes of Administration

In the delivery method of the present invention, tissues comprising neural pathways associated with the olfactory or trigeminal nerve are contacted with a composition comprising a polynucleotide agent, such as a chimeric or mixed-backbone oligonucleotide. In the context of this invention, the terms “to contact” or “contacting” a tissue with a composition means to physically apply that composition, in a form that is appropriate for the type of in vivo tissue to which the agent is being applied. For therapeutic use, a method of inhibiting cellular utilization of a mRNA encoding a target protein the expression of which is known to contribute to the pathology of a CNS disorder or disease and a method of regulating a biological activity of a target protein are provided. In general, for a therapeutic use, a patient known to require such therapy is administered a polynucleotide agent in accordance with the delivery method of the invention, possibly in a pharmaceutically acceptable carrier, in amounts and for periods of time that will vary depending upon the nature of the particular disease, its severity, and the patient's overall condition. The formulation of therapeutic compositions for administration to a particular tissue or site according to the method of the invention is believed to be within the skill in the art having access to the instant disclosure.

Nasal Cavity Administration

In one embodiment, the invention provides a method for the delivery of polynucleotide agents to the CNS by way of a neural pathway (e.g., a trigeminal or olfactory neural pathway) subsequent to intranasal administration. This embodiment of the invention can accomplish delivery of the agent the brain stem, cerebellum, spinal cord, and cortical and subcortical structures. The agent alone may facilitate this movement into the CNS, brain, and/or spinal cord. Alternatively, the carrier or other transfer-promoting factors may assist in the transport of the agent into and along the trigeminal and/or olfactory neural pathway. Administration of a therapeutic agent to the nasal cavity of a therapeutic allows the agent to bypass the BBB and travel directly from the nasal mucosa and/or epithelium to the brain and spinal cord.

Upon administration to the nasal cavity, delivery via either the olfactory or trigeminal neural pathway may employ movement of the agent through the nasal mucosa and/or epithelium to reach the nerve or a perivascular and/or lymphatic channel that travels with the nerve. Delivery by way of a neural pathway may employ movement of an agent through the nasal mucosa and/or neuroepithelium to reach the nerve or a perivascular and/or lymphatic channel that travels with the nerve.

For example, the polynucleotide agent can be administered to the nasal cavity in a manner that employs extracellular or intracellular (e.g., transneuronal) anterograde or retrograde transport into and along the olfactory and/or trigeminal nerve to reach the brain, the brain stem, or the spinal cord. Once the agent is dispensed into or onto nasal mucosa and/or epithelium innervated by the olfactory and/or trigeminal nerve, the agent may transport through the nasal mucosa and/or epithelium and travel along neurons into areas of the CN'S including the brain stem, cerebellum, spinal cord, olfactory bulb, and cortical and subcortical structures.

Alternatively, administration to the nasal cavity can result in delivery of a polynucleotide agent into a blood vessel perivascular space or a lymphatic that travels with the trigeminal and/or olfactory nerve to the pons, olfactory bulb, and other brain areas, and from there into meningeal lymphatics associated with portions of the CNS such as the spinal cord. Transport along the trigeminal and/or olfactory nerve may also deliver agents administered to the nasal cavity to the olfactory bulb, midbrain, diencephalon, medulla, cortical and subcortical structures and to the spinal cord and cerebellum. An agent administered to the nasal cavity can enter the ventral dura of the brain and may travel in lymphatic channels within the dura.

In addition, the method of the invention can be carried out in a way that employs a perivascular pathway and/or an hemangiolymphatic pathway, such as a lymphatic channel running within the adventitia of a cerebral blood vessel, to provide an additional mechanism for transport of a polynucleotide agent to the brain and/or spinal cord from the nasal mucosa and/or epithelium. An agent transported by the hemangiolymphatic pathway does not necessarily enter the circulation.

Transdermal and Sublingual Administration

In other embodiments, the method of the invention can employ delivery of a polynucleotide agent by way of a neural pathway, e.g., a trigeminal neural pathway, after transdermal (i.e., through or by way of the skin) or sublingual (applied to the underside of the tongue) administration. Upon transdermal or sublingual administration, delivery via the trigeminal neural pathway may employ movement of an agent through the skin or from under the tongue and across the lingual epithelium to reach a trigeminal nerve or a perivascular and/or lymphatic channel that travels with the nerve.

For example, a polynucleotide agent can be administered transdermally or sublingually in a manner that employs extracellular or intracellular (e.g., transneuronal) anterograde and retrograde transport into and along the trigeminal nerve pathway to reach the brain, the brain stem, or the spinal cord. Once dispensed into or onto (i.e., contacted with) skin innervated by the trigeminal nerve, or under the tongue, the agent may transport through the skin or under the tongue and across the lingual epithelium, respectively, and travel along trigeminal neurons into areas of the CNS including the brain stem, cerebellum, spinal cord, and cortical and subcortical structures. Alternatively, transdermal or sublingual administration can result in delivery of an agent into a blood vessel perivascular space or a lymphatic that travels with the trigeminal nerve to the olfactory bulb, pons, and other brain areas, and from there into meningeal lymphatics associated with portions of the CNS such as the spinal cord. Transport along the trigeminal nerve may also deliver transdermally or sublingually administered agents to the midbrain, diencephalon, medulla, and cerebellum. The ethmoidal branch of the trigeminal nerve enters the cribriform region. A transdermally or sublingually administered agent can enter the ventral dura of the brain and may travel in lymphatic channels within the dura.

In addition, the method of the invention can be carried out in a way that employs a perivascular pathway and/or an hemangiolymphatic pathway, such as a lymphatic channel running within the adventitia of a cerebral blood vessel, to provide an additional mechanism for transport of the polynucleotide agent to the spinal cord from the skin or from underneath the tongue. An polynucleotide agent transported by the hemangiolymphatic pathway does not necessarily enter the circulation. Blood vessel lymphatics associated with the circle of Willis as well as blood vessels following the trigeminal nerve can also be involved in the transport of the agent.

Transderinal or sublingual administration employing a neural pathway can deliver a polynucleotide agent to the brain stem, cerebellum, spinal cord, and cortical and subcortical structures. The agent alone may facilitate this movement into the CNS, brain, and/or spinal cord. Alternatively, the carrier or other transfer-promoting factors may assist in the transport of the agent into and along the trigeminal neural pathway. Transdermal or sublingual administration of a therapeutic agent can bypass the BBB through a transport system from the skin to the brain and spinal cord.

Disorders of the Centraol Nervous System

The present method can be employed to deliver polynucleotide agents to the brain for the treatment or prevention of disorders or diseases of the CNS, including the brain and/or spinal cord. The term “treatment” as used herein refers to reducing or alleviating symptoms in a subject, preventing symptoms from worsening or progressing, inhibition or elimination of the causative agent, or prevention of the infection or disorder in a subject who is free therefrom. Thus, for example, treatment of a cancer patient can result in reduction of tumor size, elimination of malignant cells, prevention of metastasis, or the prevention of relapse in a patient who has been cured. Treatment of infection includes destruction of the infecting agent, inhibition of or interference with its growth or maturation, neutralization of its pathological effects, and the like.

As used herein the term “central nervous system disorders” encompasses disorders and disease of the brain and/or spinal cord and includes disorders that are either neurologic or psychiatric. For example, the term includes, but is not limited to, disorders involving neurons, and disorders involving glia, such as astrocytes, oligodendrocytes, ependymal cells, and microglia; cerebral edema; raised intracranial pressure, and herniation; infections, such as acute meningitis, including acute pyogenic (bacterial) meningitis and acute aseptic (viral) meningitis, acute focal suppurative infections, including brain abscess, subdural empyema, and extradural abscess, chronic bacterial meningoencephalitis, including tuberculosis and mycobacterioses, neurosyphilis, and neuroborreliosis (Lyme disease), viral meningoencephalitis, including HIV-1 meningoencephalitis (subacute encephalitis), fungal meningoencephalitis, and other infectious diseases of the nervous system; transmissible spongiform encephalopathies (prion diseases); demyelinating diseases, including multiple sclerosis, multiple sclerosis variants, acute disseminated encephalomyelitis and acute necrotizing hemorrhagic encephalomyelitis, and other diseases with demyelination; degenerative diseases, such as degenerative diseases affecting the cerebral cortex, including Alzheimer disease, dementia with Lewy bodies and Pick's disease, degenerative diseases of basal ganglia and brain stem, including Parkinsonism, idiopathic Parkinson disease (paralysis agitans), progressive supranuclear palsy, corticobasal degeneration, multiple system atrophy, including striatonigral degeneration, Shy-Drager syndrome, and olivopontocerebellar atrophy, and Huntington disease; spinocerebellar degenerations, including spinocerebellar ataxias, including Friedreich ataxia, and ataxia-telanglectasia, degenerative diseases affecting motor neurons, including amyotrophic lateral sclerosis (motor neuron disease), bulbospinal atrophy (Kennedy syndrome), and spinal muscular atrophy, diseases associated with aging, for example anosmia; seizure disorders, for example epilepsy; tumors, such as gliomas, including astrocytoma, including fibrillary (diffuse) astrocytoma and glioblastoma multiforme, pilocytic astrocytoma, pleomorphic xanthoastrocytoma, and brain stem glioma, oligodendroglioma, and ependymoma and related paraventricular mass lesions, neuronal tumors, poorly differentiated neoplasms, including medulloblastoma, other parenchymal tumors, including primary brain lymphoma, germ cell tumors, and pineal parenchymal tumors, meningiomas, metastatic tumors, paraneoplastic syndromes, peripheral nerve sheath tumors, including schwannoma, neurofibroma, and malignant peripheral nerve sheath tumor (malignant schwannoma), and neurocutaneous syndromes (phakomatoses), including neurofibromatosis, including Type 1 neurofibromatosis (NF1) and Type 2 neurofibromatosis (NF2); affective disorders (e.g., depression and mania) anxiety disorders, obsessive compulsive disorders, personality disorders, attention deficit disorder, attention deficit hyperactivity disorder, Tourette Syndrome, Tay Sachs, Nieman Pick, and other lipid storage and genetic brain diseases, schizophrenia, and/or a prion disease.

In an alternative embodiment, the method can also be employed in subjects suffering from, or at risk for, nerve damage from a cerebrovascular disorder such as stroke in the brain or spinal cord, from CNS infections including meningitis and HIV, and/or from tumors of the brain and spinal cord. The method can also be employed to deliver polynucleotide agents to counter CNS disorders resulting from ordinary aging (e.g., anosmia or loss of the general chemical sense), brain injury, or spinal cord injury.

Pathological changes (e.g., degeneration) have been observed in the olfactory mucosa and olfactory bulb as well as other brain regions connected with the olfactory bulb of individuals afflicted with Alzheimer's Disease (AD). Therefore, the method of the invention may be particularly beneficial for the treatment of AD.

Target Sequences

Antisense agents complementary to a nucleotide (DNA or RNA) sequence of a target sequence specific for a cell growth factor, cell growth factor receptor, cytokine, cytokine receptor, seven transmembrane domain receptor (e.g., GCPR), enzyme, transcription factor, or other protein known to play a role in a CNS disorder or disease can be delivered to the CNS according to the method of the invention. Accordingly, antisense agents can be designed to be complementary to the nucleic acid sequence of target genes encoding a protein selected from, but not limited to: a tumor suppressor (e.g., p53); a transcription factor (e.g., c-jun, c-fos, jun-B); a receptor tyrosine kinase; an amyloid precursor protein; a protein kinase (e.g., tau protein kinase I); a cell cycle regulating factor (e.g., cdc-25); a protease (e.g., a cysteine protease such as CHM-1); serpine; an enzyme (e.g., steroid hydroxylase, acetylcholine hydrolyzing enzyme); an RNA editing enzyme; a growth factor (e.g., nerve growth factor, IGF-1); a G-protein coupled receptor or a cytokine receptor (e.g., the insulin-like growth factor receptor I (IGF-IR).

For example, an appropriate antisense agent for preventing the growth of a solid tumor can comprise a sequence designed to specifically hybridize with a nucleotide sequence for a cell growth factor gene, a G-protein coupled receptor gene, or a cell growth factor receptor gene. Accordingly, an antisense sequence complementary to the insulin-like growth factor receptor I (IGF-IR) gene, the insulin-like growth factor-I (IGF-I) gene, the insulin-like growth factor II (IGF-II) gene, or the platelet derived growth factor (PDGF) gene may be delivered according to the method of the invention. Antisense sequences complementary to gene sequences for one or more of these factors or receptors could be used either alone or in combination. Alternatively, an antisense composition for use in the method of the invention may comprise more than one antisense agent having sequence-specificity for the same endogenous target sequence. For example, a composition could comprise two or more chimeric or mixed-backbone oligonucleotides each designed to be complementary to a distinct region of the target sequence.

In the case of an antisense agent that is specific for a growth factor receptor gene that can be administered to prevent tumor cell growth, an antisense agent specific for the IGF-IR gene may be administered. It has been demonstrated that the in vitro expression of an antisense RNA to the endogenous IGF-IR mRNA in a rat glioblastima cell has both abrogated tumorigenesis and mediated regression of established wild-type tumors in syngeneic rats. Resnicoff et al. (1984) Cancer Res. 54:2218-2222; Resnicoff et al. (1994) Cancer Research 54:4848-4850. More specifically, in the case of an antisense sequence useful against IGF-IR, a suitable agent may be designed to be complementary to a sequence selected from the following nonlimiting mammalian IGF-IR target sequences: the polynucleotide comprising codons 1-309 of the open reading frame of the IGF-IR sequence presented in U.S. Pat. No. 5,714,170, the teachings of which are hereby incorporated by reference; a contiguous portion (fragment) of the nucleotide sequence comprising the open reading frame of a mammalian IGF-IR gene; and a noncoding region of the nucleotide sequence of a mammalina IGF-IR gene. It is be understood that an oligonucleotide sequence that comprises mismatches within the oligonucleotide sequence relative to the endogenous target sequence, which achieves the methods of the invention, such that the mismatched sequences are sufficiently complementary to the target sequence to participate in specific hybridization are also contemplated by this definition of antisense agent.

Polyuvleotide Agents Comprising Coding Sequences

The polynucleotide agent delivered/administered to the cells and tissues of the CNS can take any number of forms, and the present invention is not limited to any particular polynucleotide coding for any particular polypeptide or to any particular target protein selected for antisense-mediated inhibition. Plasmids containing genes coding for a large number of physiologically active peptides or proteins implicated in the pathology of diseases and disorders of the CNS have been reported in the literature and can be readily obtained by those of skill in the art. For example, the encoded polypeptide can comprise a peptide that encodes a biologically active portion or fragment of a protein. In preferred embodiments of this invention, the polypeptide may be an enzyme, a hormone, a growth factor or a regulatory protein.

In one embodiment of the invention, a polynucleotide agent suitable for use in the delivery method of the invention may code for therapeutic polypeptides, and these sequences may be used in association with other polynucleotide sequences coding for regulatory proteins that control the expression of these polypeptides. The regulatory protein can act by binding to genomic DNA so as to regulate its transcription; alternatively, it can act by binding to messenger RNA to increase or decrease its stability or translation efficiency.

Also provided by the present invention is a method for treating a disease or disorder of the CNS mediated by the deficiency or absence of a functional polypeptide in a mammal comprising the step of, introducing a composition comprising a naked polynucleotide sequence operatively coding for the polypeptide into a recipient and permitting the polynucleotide to be incorporated into cells of the CNS, wherein the polypeptide is formed as the translation product of the polynucleotide and the deficiency or absence of the polypeptide is effectively treated.

Diseases which result from deficiencies of critical proteins may be appropriately treated by introducing into specialized cells, DNA or mRNA coding for these proteins. A variety of growth factors such as nerve growth factor and fibroblast growth factor have been shown to affect neuronal cell survival in animal models of Alzheimer's disease. For example, cholinergic activity is diminished in patients with Alzheimer's and the expression of transduced genes expressing growth factors in the brain tissue of an afflicted patient could reverse the lost of function of specific neuronal groups.

In addition, the critical enzymes involved in the synthesis of other neurotransmitters such as dopamine, norepinephrine, and GABA have been cloned and are available. The critical enzymes could be locally increased by gene transfer into a localized area of the brain. The delivery method of the invention could be utilized to provide polynucleotide sequences to facilitate expression of the enzymes responsible for neurotransmitter synthesis. For example, the gene for choline acetyl transferase could be expressed within the brain cells (neurons or glial) of specific areas to increase acetylcholine levels and improve brain function. The increased productions of these and other neurotransmitters would have broad relevance to manipulation of localized neurotransmitter function and thus to a broad range of brain disease in which disturbed neurotransmitter function plays a crucial role.

It is well known that DNA-based gene-transfer protocols require the use of a polynucleotide sequence engineered to include appropriate signals for transcribing (promoters, enhancers) and processing (splicing signals, polyadenylation signals) the mRNA transcript. For example, a T7 polymerase gene can be used in conjunction with a gene of interest to obtain an effect of longer duration. Episomal DNA such as that obtained from the origin of replication region for the Epstein Barr virus can be used, as well as that from other origins of replication that are functionally active in mammalian cells, and preferably those that are active in human cells. Episomal DNA, for example, could be active for a number of weeks and possibly months, and cyclic administration would only be necessary upon notable regression by the patient.

Where the polynucleotide agent is a DNA molecule, promoters suitable for use in various mammalian species are well known. For example, in humans a promoter such as CMV IEP may advantageously be used. Alternatively, a cell-specific promoter can also be used to permit expression of the gene only in the target cell. All forms of DNA, whether replicating or non-replicating, which do not become integrated into the genome, and which are expressible, are within the methods contemplated by the invention. In a particular embodiment of this aspect of the invention, the DNA sequence contains regulatory elements including a promoter, and still more preferably, a neuron specific promoter.

When the polynucleotide agent to be delivered according to the delivery method of the invention is mRNA, it can be readily prepared from the corresponding DNA in vitro. For example, conventional techniques utilize phage RNA polymerases SP6, T3, or T7 to prepare mRNA from DNA templates in the presence of the individual ribonucleoside triphosphates. An appropriate phage promoter, such as a T7 origin of replication site is placed in the template DNA immediately upstream of the gene to be transcribed.

One of skill in the art will recognize that embodiments of the invention that contemplate the use of a polynucleotide agent comprising a mRNA molecule also require the appropriate structural and sequence elements for efficient and correct translation, together with those elements which will enhance the stability of the transfected mRNA. In general, translational efficiency has been found to be regulated by specific sequence elements in the 5′ non-coding or untranslated region (5′ UTR) of the RNA. Positive sequence motifs include the translational initiation consensus sequence (GCC) GCCA/GCCATGG (Kozak (1987) Nucleic Acids Res. 15:8125) and the ⁵G 7-methyl GpppG cap structure (Drummond et al. (1985) Nucleic Acids Res. 13:7375. Negative elements include stable intramolecular 5′ UTR stem-loop structures (Muesing et al. (1987) Cell 48:691(1987)) and AUG sequences or short open reading frames preceded by an appropriate AUG in the 5′ UTR (Kozak, supra; Rao et al. (1988) Mol. Cell. Biol. 8:284). mRNA-based polynucleotide agents suitable for use in the delivery method of the invention disclosed herein should include appropriate 5′ UTR translational elements flanking the coding sequence for the protein of interest.

In addition to translational concerns, mRNA stability must be also be considered during the design and preparation of a mRNA-based polynucleotide agent. It is well known that capping and 3′ polyadenylation are the major positive determinants of eukaryotic mRNA stability (Drummond, supra; Ross (1988) Mol. Biol. Med. 5: 1) and function to protect the 5′ and 3′ ends of the mRNA from degradation. However, regulatory elements that affect the stability of eukaryotic mRNAs have also been defined, and therefore must be considered in the development of RNA-based polynucleotide agents. The most notable and clearly defined of these are the uridine rich 3′ untranslated region (3′ UTR) destabilizer sequences found in many short half-life mRNAs (Shaw and Kamen (1986) Cell 46:659), although there is evidence that these are not the only sequence motifs which result in mRNA destabilization (Kabnick and Housman (1988) Mol. and Cell Biol. 8:3244). In addition just as viral RNA sequences have evolved that bypass normal eukaryotic mRNA translational controls, likewise some viral RNA sequences seem to be able to confer stability in the absence of 3′ polyadenylation (McGrae and Woodland (1981) Eur. J. Biochem. 116:467).

In addition, the present invention includes the use of mRNA polynucleotide agent that is chemically modified or blocked at the 5′ and/or 3′ end to prevent access by RNase. This enzyme is an exonuclease and therefore does not cleave RNA in the middle of the chain. It is well known that if a group with sufficient bulk is added, access to the chemically modified RNA by RNAse can be prevented. Such chemical blockage can substantially lengthen the half life of the RNA in vivo. Two agents that may be used to modify RNA are available from Clonetech Laboratories, Inc., Palo Alto, Calif.: C2 AminoModifier (Catalog #5204-1) and Amino-7-dUTP (Catalog #K1022-1). These materials add reactive groups to the RNA. After introduction of either of these agents onto an RNA molecule of interest, an appropriate reactive substituent can be linked to the RNA according to the manufacturer's instructions.

It will be apparent to those of skill in the art that there are numerous methods available for the preparation of a RNA polynucleotide agent suitable for use in the delivery method of the invention. See, for example, the methods in Ausubel (1988) Current Protocols in Molecular Biology, Vol. 1 (John Wiley and Sons, New York). For example, the mRNA can be prepared in commercially available nucleotide synthesis apparatus. Alternatively, mRNA in circular form can be prepared. Exonuclease-resistant RNAs such as circular mRNA, chemically blocked mRNA, and mRNA with a 5′ cap are preferred, because oft heir greater half-life in vivo. In particular, one preferred mRNA is a self-circularizing mRNA having the gene of interest preceded by the 5′ untranslated region of polio virus. It has been demonstrated that circular mRNA has an extremely long half-life (Harland and Misher (1988) Development 102:837-852) and that the polio virus 5′ untranslated region can promote translation of mRNA without the usual 5′ cap (Pelletier and Sonnenberg (1988) Nature 334:320-325, hereby incorporated by reference).

Antisense Agents

As used herein the term “antisense agent” refers to a sequence-specific regulator (e.g., neuroregulatory) of gene expression and target protein function. Suitable antisense agents for use with the method of the invention include, but are not limited to, isolated polynucleotides, synthetic antisense oligonucleotides, antisense polynucleotides produced in vivo from an expression vector, and antisense peptide nucleic acids (PNAs). The effectiveness of an antisense agent depends upon numerous factors, including the type of cell that comprises the target mRNA or protein, the local concentration of the agent at the endogenous target mRNA or protein, the rate of synthesis and degradation of the target mRNA and its encoded protein, the accessibility of the target sequence, the specificity of the antisense agent, and the nature of the mechanism of action (e.g., inhibiting of mRNA translation, affecting RNA splicing, or inducing RNase H-mediated degradation of the target mRNA). In addition, the type of agent will also influence its characteristics and mechanism of cellular uptake. In one embodiment, the polynucleotide agent comprises a short synthetic oligonucleotide or an oligonucleotide mimic (e.g., PNA molecule) that is a sequence-specific regulator of nucleic acid utilization.

The delivery and activity of antisense agents of the invention may be assayed for activity using standard protocols. For example, one may employ the protocol demonstrated in the Examples described below to demonstrate delivery of the agent to the CNS according to the method of the invention. Agents that exhibit strong binding to receptors will be expected to exert antagonistic activity, which may be determined by means of appropriate cell-based or in vivo assays known in the art.

As used herein the terms “antisense molecule” and “antisense agent” are used interchangeably and to refer to a molecule comprising a nucleotide sequence designed, according to the rules of Watson-Crick base pairing, to be complementary to an endogenous nucleic acid (e.g., DNA or RNA) target that can hydrogen bond to the target sequence under physiologic conditions and thereby inhibit cellular utilization of the targeted nucleic acid. It is to be understood that the administration of an antisense agent ultimately regulates (e.g., modulates) the amount of target protein. This is accomplished by providing antisense agents that “specifically hybridize” with the targeted endogenous polynucleotide molecule. Generally, the target nucleic acid is an endogenous mRNA molecule.

The relationship between an antisense molecule, such as an oligonucleotide, and the complementary endogenous nucleic acid target molecule to which it hybridizes is commonly referred to as “antisense.” Accordingly, the term encompasses a native antisense polynucleotide, a synthetic antisense oligodeoxynucleotide, an antisense nucleic acid sequence produced in vivo from an expression vector, and an antisense peptide nucleic acid. For example, a suitable antisense molecule for use in the method of the invention may comprise a synthetic antisense oligodeoxynucleotide designed to be complementary to a mRNA molecule or a vector capable of directing the production of an antisense nucleotide sequence in vivo. More specifically, the present invention employs antisense agents to regulate (inhibit) the expression and/or fuiction of a target protein that is known to be associated with the pathology of a CNS disorder or disease.

Generally speaking, antisense molecules rely on the formation of Watson-Crick hydrogen bonds between the antisense agent and the complementary target nucleic acid strand to provide a high degree of specificity to their regulatory activity. As used herein the term “antisense molecule” encompasses linear oligomers of natural or modified monomers or linkages, including deoxyribonucleosides, ribonucleosides, polyamide nucleic acids, and the like, capable of specifically binding to a target polynucleotide by way of a regular pattern of monomer-to-monomer interactions (e.g., nucleoside-to-nucleoside). Generally, monomers are linked by phosphodiester bonds or analogs thereof to form oligonucleotides ranging in size from a few monomeric units, e.g., 4-8 monomers, to several hundreds of monomeric units. Ideally, the antisense molecule should not hybridize to any other nucleic acid sequence in the cell except the target sequence and should not bind nonspecifically to other cellular constituents such as proteins.

In the context of this invention, the term “hybridization” means hydrogen bonding, also known as Watson-Crick base pairing, between complementary bases, usually on opposite nucleic acid strands. Guanine and cytosine are examples of complementary bases that are known to participate in Watson-Crick base pairing by the formation of three hydrogen bonds. Adenine and thymine also exemplify complementary bases that interact to form two hydrogen bonds between them. “Specifically hybridizable” and “complementary” are terms that are used to indicate a sufficient degree of complementarity such that stable and specific binding occurs between the endogenous nucleic acid target and the antisense agent. It is understood that an oligonucleotide need not be 100% complementary to its target nucleic acid sequence to participate in specific hybridization.

In one embodiment, a suitable antisense nucleic acid molecule for use in the method of the invention can be complementary to a contiguous region of ribonucleotide sequence that comprises a portion of the coding region of a targeted mRNA. The term “coding region” is understood to refer to the portion of a mRNA sequence that consists of the codons that are translated into the amino acid sequence of a polypeptide. In an alternative embodiment, the antisense nucleic acid molecule is antisense (i.e., complementary) to a “noncoding sequence” of the targeted mRNA. The term “noncoding sequence” refers to nucleotide sequence that is not translated into amino acid sequence. It is to be understood that as used in the context of this invention, the term “mRNA” includes not only the coding region but also the flanking noncoding sequences of contiguous ribonucleotides located upstream and downstream of the coding region. These regions are known to a person of skill in the art to include the 5′-untranslated region, the 3′-untranslated region, the 5′ cap region, intron regions, and intron/exon or splice junction ribonucleotides. Thus, oligonucleotides designed in accordance with this invention can target wholly or partially these flanking ribonucleotide sequences as well as the sequence of the coding ribonucleotides.

In one embodiment, the oligonucleotide is targeted to the translation initiation site or the “start codon region” or sequences in the 5′- or 3′-untranslated region of the mRNA molecule. The terms “start codon region”, “AUG region”, and “translation initiation codon region” are used synonymously herein and refer to a portion of a mRNA or gene that encompasses from about 25 to about 50 contiguous nucleotides in either direction (i.e., 5′ or 3′) from a translation initiation codon. This region is a preferred RNA-binding site for the design of antisense agents. Other regions that may be targeted include a nucleotide sequence of the 5′-untranslated region, a potential splice site located at an intron-exon junction, a sequence located within an exon region, or a sequence located in the 3′-untranslated region.

There is substantial guidance in the literature for the design and identification of antisense regulatory agents and it is well known by one of skill in the art that a preferred antisense agent will have the following characteristics: a unique complementary sequence that is specific for an accessible target RNA-binding site; efficient cellular uptake; in vivo biological stability, and an antisense mechanism of action that successfully reduces the mRNA and/or target protein level (for example see Sezakiel et al. (2000) Frontiers in Bioscience 5:d194). Because there are no a priori rules to predict the most desirable antisense sequence, one of skill in the art will recognize the need to empirically design effective antisense agents. Accordingly, it would not be unreasonable to design at least ten different antisense sequences that are complementary to sequences contained in the targeted nucleotide sequence. One of skill in the art could readily employ the principles of Watson-Crick base pairing to design oligonucleotides that maximize hybridization while avoiding sequences with regions of polyguanosine or G-C arms that could potentially form strong hairpins. See, for example, An Antisense Oligonucleotide Primer by Richard I. Hogrete available at www.trilink.com.

In general terms, the preparation of a suitable antisense agent for use in the method of the current invention involves the steps of: (1) identifying a target sequence in a nucleic acid molecule encoding a protein that contributes to the pathology of a disorder of the CNS; (2) selecting a RNA-binding site (e.g., the start codon region) that is consistent with a particular termination mechanism; and (3) modifying the backbone of the antisense agent to confer a desirable affinity and/or in vivo stability. An advantage of selecting synthetic oligodeoxyribonucleotides as antisense agents is the simplicity of their synthesis and purification, and their amenability to high-through-put screening to identify agents capable of specific hybridization to the target sequence.

The generation or production of an antisense agent within the target cell offers an alternative to the delivery of exogenous antisense agents to the CNS. It is well known that endogenous production can be accomplished by the use of an expression plasmid or expression vector comprising a nucleotide sequence (e.g., DNA) encoding an antisense RNA. Thus in an alternative embodiment, a viral vector-mediated or nonviral vector-mediated delivery method can be used for the delivery of a nucleotide sequence encoding an sequence capable of directing the endogenous production of an antisense agent, for example an oligonucleotide. See Luo and Saltzman (2000) Nature Biotechnology 18:33.

The use of an expression vector or eukaryotic expression plasmid to generate antisense agents intracellularly (e.g., endogenously) offers several potential advantages over the exogenous administration of an antisense agent. For example, an antisense RNA that is produced in vivo can be more effectively delivered (e.g., achieve higher copy number) to specific cells and or tissues of the CNS relative to the efficiency of an exogenous delivery protocol, particularly in light of the fact that enzymatic degradation of native oligonucleotides is so prominent in vivo. Thus, the duration or residence time of the antisense agent will likely be longer when it is delivered in the context of a delivery method that facilitates endogenous production, particularly if the vector-mediated transfer of the sequence results in the sequence becoming incorporated in the genome of the recipient, but also if in vivo production occurs as a result of episomal expression. In addition, the opportunity to select a particular expression control element, such as for example, promoter sequences, affords the opportunity to accomplish tissue-specific (e.g., neuronal cell or glial cell), site-specific (e.g., nuclear or cytoplasmic), or inducible (e.g., by the administration of a transcription activator) production of the antisense agent.

Eukaryotic expression plasmids or viral vectors represent suitable vehicles for use with the antisense applications of the invention. Suitable plasmids for use with this embodiment of the invention include the nonintegrative plasmids discussed above as well as plasmids that are designed to integrate a polynucleotide sequence into the genome of a recipient cell. The choice of an appropriate vector will be dictated by the identity of the tissue or cell that is targeted for delivery. For example, because mature neurons do not divide, a retroviral vector capable of integration only into dividing cells would not be a suitable selection. However, an adenoviral or adeno-associated vector can be employed for the delivery of antisense olignucleotides (e.g. polynucleotides) described herein. In vitro studies have clearly established that neurons and glial cells in particular are highly susceptible to infection with replication defective adenoviruses. Caillaud et al. (1993) Eur. J Neurosci. 5:1287-1291. In addition, it has also been demonstrated that the direct intracerebral or intraventricular injection of a replication-defective adenoviral vector resulted in infection of neurons, glial, and epindymal cells. Davidson. et al. (1993) Nature Genetics 3:219-223; Akli et al. (1993) Nature Genetics 3:224-228. Draghia et al. have successfully utilized an adenoviral vector for the delivery of an E. coli lacZ gene to the CNS of rats after nasal instillation (Draghia et al. (1995) Gene Therapy 2:418-423.

Consistent with these observations, a viral vector can be utilized for the localized delivery of a replication-deficient adenovirus comprising a DNA sequence encoding an antisense agent. In one embodiment, a viral vector comprising a nucleotide (e.g., DNA) sequence encoding an antisense oligonucleotide agent is delivered according to the method of the invention. In a second embodiment, a viral vector comprising an antisense olignucleotide is delivered according to the methods of the invention.

The preparation of a suitable antisense agent for use in the method of the invention is a multistep process that begins with identification a nucleic acid sequence encoding a protein whose function is to be regulated. Selection of a suitable antisense sequence depends on knowledge of the nucleotide sequence of the target mRNA, or gene from which the mRNA is transcribed. For example, as discussed above in the context of an antisense sequence specific for a mammalian IGF-IR, an oligonucleotide designed to be complementary to a contiguous sequence present in signal sequence embodies a suitable antisense agent for use in the method of the invention.

The process also requires the selection of a target RNA-binding site (or sites) within the nucleic acid sequence for the oligonucleotide interaction to occur such that the desired effect, a modulation of gene expression (e.g., inhibition of mRNA processing or of translation) will occur. Once the RNA-binding site has been identified, a complementary oligonucleotide (or an oligonucleotide mimic) is designed to specifically hybridize to the endogenous nucleic acid sequence under physiologic conditions. In order to be an effective therapeutic agent binding of the antisense agent to its target sequences must interfere with the transcription or translation of the targeted DNA or mRNA in a manner that is sufficient to inhibit the intracellular level of the target protein. In general, target sequences encoding initiation sequences, termination sequences and splice regions are considered to have the potential to produce the most effective inhibition. Depending upon the type of the antisense agent, the final step required for the preparation of a suitable antisense oligonucleotide for exogenous administration may also involve the introduction of a modification into the backbone of the oligonucleotide to produce a polynucleotide analogue. In general, chemically modified antisense agents (e.g., phosphothioate or morpholino polynucleotide analogues) demonstrate increased stability to nuclear degradation compared to unmodified sequences. As a result, chemically modified oligonucleotides are more effective both in vitro and in vivo. Although targeting to mRNA is preferred and exemplified in the description below, it will be appreciated by those skilled in the art that other forms of nucleic acid, such as pre-mRNA or genomic DNA, may also be targeted.

For purposes of the present invention, the terms “polynucleotide analogue” and “oligonucleotide” are used interchangeably herein and connote oligomers (polymers) of natural (e.g., native) or modified monomers or linkages, including deoxyribonucleosides, ribonucleosides, polyamide nucleic acids, and the like, capable of specifically binding to a target polynucleotide sequence by way of a regular pattern of monomer-to-monomer interactions (e.g., nucleoside-to-nucleoside), thereby altering the intermediary metabolism of mRNA. The resulting complex is stabilized by hydrogen bonding, which can mediated by Watson-Crick base pairing, Hoogstein binding, or any other sequence-specific maimer of binding. Usually, monomers are linked by phosphodiester bonds or analogs thereof to form oligonucleotides ranging in size from a few monomeric units, e.g., 3-4, to several hundreds of monomeric units. The sequence of nucleotides may be interrupted by non-nucleotide components. More specifically, as used herein the term “oligonucleotide” includes single-stranded oligonucleotides composed of naturally occurring nucleobases, sugars, and covalent intersugar (backbone) linkages as well as oligonucleotides having non-naturally occurring (e.g., modified) backbones that function similarly. Thus, as used herein the term “oligonucleotide” encompasses natural oligomers and the chemical analogs and chimeric molecules described below.

Delivery of a modified or substituted oligonucleotide according to the method of the invention may be preferable to the delivery of a native oligomer because the modification could confer a desirable property such as, for example, enhanced binding to the targeted polynucleotide or resistance to nuclease degradation. Agrawal et al. (1997) Proc. Natl. Acad. Sci. USA 94(6):2620, and Proc. Natl. Acad. Sci. USA 94(6):2620. If present, modifications to the nucleotide can be introduced either before or after assembly of the polymer. For example, an antisense agent (antisense oligonucleotide) suitable for use in the method of the invention can be chemically synthesized using naturally occurring nucleotides or various modified nucleotides or monomers.

Suitable oligonucleotides for use in the delivery method of the invention should be of sufficient length to specifically hybridize to their target nucleotide sequence and to modulate the information transfer from a gene to a protein (e.g., inhibition of translation, or splicing). The binding of an oligodeoxynucleotide to the target nucleic acid sequence may inhibit the interaction of the nucleic acid with other nucleic acids or proteins required for cellular utilization of the mRNA transcript. Appropriate oligonucleotides preferably comprise from about 8 to about 50 monomers (e.g., nucleobases). It is known in the art, that a nucleoside is a base-sugar combination in which a heterocyclic base (e.g., a purine or a pyrimidine) normally comprises the base component of the combination. Particularly preferred are antisense oligonucleotides comprising from about 10 to about 30 nucleobases (i.e., from about 10 to about 30 linked nucleosides). Nucleotides are nucleosides that further include a phosphate group covalently linked to the sugar portion of the nucleoside. In the context of antisense molecules that comprise non-naturally occurring monomeric units, it is to be understood that suitable antisense agents comprise 8 to 50 monomers. Accordingly, suitable antisense oligonucleotides may be of any suitable length, e.g., from about 10 to 50 nucleotides in length (e.g., 10, 12, 14, 15, 17, 20, 25, 30, 35, 40, 45 or 50 nucleobases or monomers) and may contain phosphorothioates, phosphotriesters, methylphosphonates, short chain alkyl or cycloalkyl intersugar linkages, or short chain heteroatomic or heterocyclic intersugar (“backbone”) linkages. However, it should be noted that a higher in vivo intracellular concentration of the antisense agent is more likely to be achieved with the use of a relatively small (e.g., less than 12 nucleobases) oligonucleotide because of a higher efficiency of uptake by cells in vivo. It is well known that an antisense oligonucleotide comprising 13-15 complementary nucleotides is statistically predicted to bind to a single sequence. Preferably, antisense oligonucleotides should be at least 15 nucleotides long, to achieve adequate specificity. In a preferred embodiment, a 20-nucleotide antisense molecule is utilized.

Although a number of potential cell surface receptors for oligonucleotides have been described (including the MAC-I intergrin, scavenger receptors, and a protein that may act as an oligonucleotide transporter), it appears as if the majority of oligonucleotides are taken up by endocytosis and as a consequence tend to initially accumulate in an endosomal-lysososomal compartment. More specifically, it is believed that the internalization of oligonucleotides predominantly depends on adsorptive endocytosis and pinocytosis (fluid-phase endocytosis). The role of the active process of adsorptive endocytosis is suggested by the observation that charged oligonucleotides (i.e., phosphorodiesters and phosphorothioates) that are known to adsorb to cell surfaces are internalized to a much higher level than uncharged oligonucleotides (e.g., peptide nucleic acids or methyl phosphonates). Pinocytosis is a constitutive cellular process in which cells engulf water and solutes dissolved therein, and in situations of relatively high local oligonucleotide concentration offers an alternative method of internalization.

Numerous mechanisms have been proposed to explain how an antisense oligonucleotide regulates the activity of its target mRNA, including the inhibition of the processing of the primary RNA transcript (e.g., capping, methylation, splicing, 3′-polyadenylation), inhibition of mRNA transport out of the nucleus, and inhibition of translation (e.g., cellular utilization) by hybridization arrest. Alternatively, an oligodeoxynucleotide can activate the destruction of the target mRNA by an RNase H—dependent mechanism. Although the mechanism of action of antisense oligonucleotides may differ from cell type to cell type and may vary depending on the nature of the endogenous nucleotide sequence that is targeted for binding, there is strong evidence that the predominant mechanism of action in vitro is mediated by the enzymatic cleavage of the target RNA by RNase H. Dash et al. (1987) Proc. Natl. Acad. Sci. USA 84:7896-7900; Walder and Walder (1988) Proc. Natl. Acad. Sci. USA 85:5011-5015. RNase H is a ubiquitous enzyme that specifically degrades the RNA strand of an RNA-DNA heteroduplex (i.e., hybrid). It is well known that RNase H enzymes do not require long hybrid regions as substrates; thus it is not possible to increase the specificity of an antisense agent by increasing the length of the oligonucleotide. It has been estimated that as few as ten base pairs are likely to be sufficient in human cells. Branch (1998) Trends Biochem Sci. 23(2):45-50.

It is well known that cells contain a variety of endo- and exonucleases and that oligonucleotides in their natural form are subject to rapid enzymatic digestion in vivo. Accordingly, the major route of oligonucleotide elimination in vivo appears to be via their enzymatic degradation. In one embodiment, the antisense agent is an antisense oligonucleotide that is modified to improve the biophysical, biochemical, pharmacokinetic, or safety profile of a native phosphodiester oligonucleotide. A number of nucleotide and nucleoside modifications have been shown to make the oligonucleotide into which they are incorporated relatively more resistant to nuclease degradation. Phosphodiester nucleotides were initially studied in cell free systems and in vitro cell cultures, however as a class of molecule they are not very stable against nucleases and therefore have limited potential as in vivo agents. In an alternative embodiment, an oligonucleotide is modified to enhance its inherent nuclease resistance. Improved nuclease stability confers favorable changes in the in vivo stability and biodistribution of the polynucleotide analogue. Accordingly, chemical analogues that are suitable for use according to the method of the present invention include, but are not limited to, analogues in which, for example, the phosphodiester bonds have been modified (e.g., to a methylphosphonate, a phosphotriester, a phosphorothioate, a phosphorodithioate, or a phosphoramidate) so as to render the oligonucleotide more stable in vivo. For example, oligodeoxyribonucleotide phosphorothioates (e.g., where one of the phosphate oxygen atoms not involved in the phosphate bridge is replaced by a sulphur atom) or oligodeoxyribonucleotide methylphosphonates (e.g., in which a nonbridging oxygen atom at the phosphorous is replaced with a methyl group) embody common chemical analogues that impart improved stability with respect to nuclease degradation. See Cohen ed. (1989) Oligodeoxnucleotides: Antisense Inhibitors of Gene Expression (CRC Press, Inc., Boca Raton, Fla.). The half-life of a phosphodiester oligomer introduced into the peripheral circulation of a mouse is about 1 minute, while the half-life of a phosphothioate oligomer is about 48 hours. (Agrawal et al. (1991) Proc. Natl. Acad. Sci. USA 88:7595.

However, it should be noted that there are some problems with the in vivo use of phosphorothioate oligonucleotides. For example, the backbone is chiral, resulting in a racemic mixture of 2ⁿ oligonucleotide species (where n=number of phosphorothioate internucleotide linkages) instead of a single compound. Furthermore, the binding affinity of a phosphothioate oligomer is lower than the affinity of its corresponding phosphodiester oligonucleotide (Agrawal et al. (1998) Antisense & Nucleic Acid Drug Dev. 8:135; LaPlanche et al. (1986) Nucleic Acids Res. 14:9081-9093). In addition, because they are negatively charged phosphorothioate oligonucleotides have been known to bind nonspecifically to cellular proteins, lipids, and carbohydrates, which can consequently mediate non-antisense effects that can result in toxicicy or which can be mistakenly attributed to an antisense effect. Phosphorothioates also have a reputation for being toxic although that may be a sequence specific phenomenon or due to contamination in early oligonucleotide preparations (Srinivasan and Iverson (1995) J. Lab. Anal. 9:129-137). In addition, the administration of phosphorothioate oligonucleotides comprising particular sequences and structural motifs has been reported to have undesirable immunostimulatory effects.

Preferred modified oligonucleotide backbones (e.g. polynucleotide analogues) that do not include a phosphorus atom therein have backbones that are formed by short chain alkyl or cycloalkyl internucleoside linkages, mixed heteroatom and alkyl or cycloalkyl intemucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene-containing backbones; sulfamate backbones; methyleneimino and methylenehydrazino backbones; sulfonate and sulfonamide backbones; amide backbones; and others having mixed N, O, and S component parts. For example, morpholino oligomers are a class of chemically modified oligonucleotides in which the ribose moiety is replaced with a morpholino group (U.S. Pat. No. 5,185,444, the teachings of which are incorporated herein by reference). The morpholino modification renders an oligomer resistant to enzymatic degradation and morpholino antisense nucleotides have been successfully utilized to inhibit the production of target proteins (e.g., TNF-α) in vivo. See Qin et al. (2000) Antisense &Nucleic Acid Drug Dev. 10:11. Nuclease resistance is routinely measured by incubating oligonucleotides with isolated nuclease solutions or cellular extracts and determining (e.g., by gel electrophoresis) the extent of intact oligonucleotide remaining over time. Oligonucleotides that have been modified to enhance their nuclease resistance survive intact for a longer time relative to the native oligonucleotides.

Appropriate antisense oligonucleotides for use with the method of the invention also include “chimeric oligonucleotides.” As used herein the term “chimeric oligonucleotide” connotes a mixed-backbone polynucleotide analogue that comprises a mixture of different sugar and/or backbone chemistries. These oligonucleotides typically contain at least one region of modified nucleotides that confers one or more beneficial properties (such as, for example, increased nuclease resistance or increased binding affinity for the RNA target) and a region that is a substrate for RNase H cleavage. The most common chimeric oligonucleotides are also referred to as “second generation” oligonucleotides. This nomenclature derives from the fact that phosphorothioates are usually considered to be the first generation antisense agents.

Chimeric, or mixed-backbone oligonucleotides vary considerably in their specific construction, but generally all of them have the same basic design characteristics; a phosphodiester or phosphorothioate central region surrounded by nuclease resistant arms. More specifically, a chimeric or mixed-backbone suitable for use in the delivery method of the invention may comprise phosphorothioate segments at the 5′ and 3′ ends and have a modified oligodeoxynucleotide or oligoribonucleotide segment located in the central portion of the oligomer. See Agrawal et al. (1997) Proc. Natl. Acad. Sci. USA 94(6): 2620. The art teaches that a good starting point is to use an oligonucleotide eighteen nucleotides in length that has six 2′-OMe nucleotides at both the 5′ and 3′ ends, leaving a core of six 2′-deoxyribose nucleosides with phosphorothioate intemucleotide linkages (Monia et al. (1996) Nat. Med. 2:668-675). The arms may or may not contain phosphorothiate linkages. Removal of phosphorothiate linkages is favorable from the point of view that it may reduce toxicity, however it will also reduce nuclease resistance. The underlying principles driving the design of a suitable chimeric oligonucleotide suitable for use in the methods of the invention are two fold: increased stability and retention of RNase H activity. Many of the chimeric oligonucleotides reported in the literature have improved properties compared to the properties of phosporothioate oligomers with respect to affinity for RNA, RNase H activation, and pharmacokinetic profiles.

Alternatively, other molecular designs that depend on extreme hybridization enhancement using highly modified oligonucleotides such as 2′-MOEs (Monia (1997) Ciba Found. Symp. 209:107-123), N3→P5′ phosphoramidates (Gryaznov and Chen (1994) J. Amer. Chem. Soc. 116:3143-3144; Mignet and Gryaznov (1 998) Nucleic Acids Res. 26:431-438), PNA's (Hanvey et al. (1992) Science 258:1481-1485), chirally pure methylphosphonates (Reynolds et al. (1996) Nucleic Acids Res. 24:4584-4591), and MMIs (Morvan et al. (1996) J. Amer. Chem. Soc. 118:255; Swayze (1997) Nucleosides Nucleotides 16:971-972) represent alternative embodiments which may be particularly useful for the inhibition of protein expression by hybrid arrest. In one embodiment, a chimeric oligonucleotide suitable for use in the method of the invention comprises at least one region modified to increase target binding affinity, and, usually, a region that acts as a substrate for RNase H. A common design is to have nuclease resistant arms (such as 2′-O-methyl (Ome) nucleosides) surrounding a phospodiester- or phosphorothioate-modified central core region (Agrawal and Goodchild (1987) J. Tetrahedran Letters 28:3539-3542; Giles and Tidds (1992) Nucleic Acid Res. 20:753-770.

In one embodiment of the invention, the antisense oligonucleotide for use in the delivery method of the invention is a chimeric antisense oligonucleotide that exhibits high resistance to endo- and exonucleases, high sequence specificity, and the ability to activate RNAse H, as evidenced by efficient and long-lasting knockout of target mRNA. See the antisense constructs described in the examples disclosed herein. Also see International Publication No. WO 01/16306 A2; and U.S. application Ser. Nos. 60/151,246, filed Aug. 27, 1999, and 09/648,254, filed Aug. 25, 2000, both entitled “Chimeric Antisense Oligonucleotides and Cell Transfecting Formulations Thereof,” the contents of which are herein incorporated by reference.

The antisense molecules of the present invention include bioequivalent compounds, including but not limited to pharmaceutically acceptable salts. “Pharmaceutically acceptable salts” are physiologically and pharmaceutically acceptable salts of the nucleic acids of the invention, i.e., salts that retain the desired biological activity of the parent compound and do not impart undesired toxicological effects thereto (see, for example, Berge et al. (1977) J. Pharma. Sci. 66:1-19). Administration of pharmaceutically acceptable salts of the polynucleotides described herein is included within the scope of the invention. Such salts may be prepared from pharmaceutically acceptable non-toxic bases including organic bases and inorganic bases. Salts derived from inorganic bases include sodium, potassium, lithium, ammonium, calcium, magnesium, and the like. Salts derived from pharmaceutically acceptable organic non-toxic bases include salts of primary, secondary, and tertiary amines, basic amino acids, and the like. For a helpful discussion of pharmaceutical salts, see Berge et al. (1977) J. Pharma. Sci. 66:1-19, the disclosure of which is hereby incorporated by reference.

For oligonucleotides, examples of pharmaceutically acceptable salts include but are not limited to (a) salts formed with cations such as sodium, potassium, ammonium, magnesium, and calcium; (b) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, and the like; (c) acid addition salts formed with inorganic acids, for example hydrochloric acid, hydrobromic acid, phosphoric acid, nitric acid, and the like; and (d) salts formed from elemental anions such as chlorine and bromine.

There is substantial guidance in the literature for selecting particular sequences for complementary oligonucleotides given a knowledge of the sequence of the target polynucleotide and the accessibility of the binding site. See, for example, Ulmann et al. (1990) Chem. Rev. 90:543-584; Crooke (1992) Ann. Rev. Pharmacol. Toxicol. 32:329-376; and Zamecnik and Stephenson (1974) Proc. Natl. Acad. Sci. USA 75:280-284. Preferably, the synthetic oligonucleotide sequence is designed so that the G-C content is at least 60%. Oligonucleotides suitable for use in the method of the invention may be conveniently and routinely produced and purified using chemical synthesis, enzymatic ligation reactions and purification procedures that are well known in the art. Equipment for such synthesis is sold by several vendors including Applied Biosystems. In general, antisense agents can comprise from about 10 to about 50 nucleotides (or monomers), preferably from about 14 to about 25 nucleotides, and more preferably from about 17 to 20 nucleotides. For example, a suitable IGF-IR antisense oligonucleotide can include, but is not limited to, a modified chimeric oligonucleotide or PNA based on a sequence selected from: TCTTCCTCACAGACCTTCGGGCAAG (SEQ ID NO: 1); TCCTCCGGAGCCAGACTT (SEQ ID NO: 2); GGACCCTCCTCCGGAGCC (SEQ ID NO: 3); CCGGAGCCAGACTTCAT (SEQ ID NO:4); CTGCTCCTCCTCTAGGATGA (SEQ ID NO:5); CCCTCCTCCGGAGCC (SEQ ID NO:6); TACTTCAGACCGAGGCC (SEQ ID NO:7); CCGAGGCCTCCT CCCAGG (SEQ ID NO:8); and TCCTCCGGAGCCAGACTT (SEQ ID NO: 9). See the examples disclosed herein and International Publication No. WO 01/16306 A2. Also see U.S. Pat. No. 5,714,170, and U.S. application Ser. Nos. 60/151,246, filed Aug. 27, 1999, and 09/648,254, filed Aug. 25, 2000, both entitled “Chimeric Antisense Oligonucleotides and Cell Transfecting Formulations Thereof,” herein incorporated by reference in their entirety.

It should also be understood that the method of the invention contemplates the administration of both exogenous single-stranded nucleotide sequences (or peptide nucleic acid oligomers) as well as antisense oligonucleotides produced in vivo from an expression vector comprising a translational unit that encodes a sequence that is complementary to a contiguous region of the target gene mRNA, which is delivered to the CNS according to the method of the invention.

Peptide Nucleic Acid (PNA) Agents

As used herein the terms “peptide nucleic acids” or “PNAs” refer to polynucleotide mimics in which the deoxyribose phosphate backbone is replaced by a pseudopeptide backbone to which the four native nucleobases are linked. More specifically, the phosphodiester backbone of DNA or RNA is replaced by a homomorphous backbone consisting of (N-2 aminoethyl) glycine units bearing nucleobases attached via methylenecarbonyl linkers. (Nielsen et al. (1991) Science 254: 1497; Larsen et al. (1999) Biochem. Et Biophysica Acta 1489:159-166). The nucleobases are maintained to mediate sequence-specific hybridization with the targeted endogenous nucleic acid target molecule. Chemically, PNA agents have a homomorphous, charge neutral, achiral polyamide backbone that is relatively flexible (Larsen et al. (1999) Biochem. Et Biophysica Acta 1489:159-166). The uncharged nature of the PNA oligomer enhances the stability of the hybrid PNA/DNA (mRNA) duplex. Accordingly, PNA agents embody a DNA mimic that is only remotely chemically related to DNA. Although PNA agents are in fact more closely related to proteins (peptides) than to nucleic acids, they provide alternative sequence-specific regulators of nucleic acid function. The method of the present invention provides an effective delivery method that could facilitate the evaluation and development of these polynucleotide mimics.

As discussed above, the antisense effect of conventional oligonucleotides and their chemical analogs rely on the activation of RNase H. However it is well known that morpholino-mRNA complexes and PNA-mRNA complexes are not substrates for RNase H activity. Thus, the proposed mechanism of action of morpholino oligomers and PNA molecules is believed to be translation arrest due to steric interference with assembly or progression of the translation machinery. PNA oligomers have been successfully used to inhibit target protein expression at both the transcriptional and translocational level. More specifically, PNA oligomers that are complementary to nucleotide sequences present at the translation start site of 5′-untranslated regions of targeted mRNA sequences have been shown to efficiently inhibit translation both in vitro and in vivo (Pooga et al. (1998) Nature Biotechnology 16:857). Appropriate target regions for PNAs reside both within and outside of the AUG region, and that the identification of suitable PNA targets will likely require fairly extensive experimentation requiring a empirical determination of an optimal target based on the results obtained from mRNA walks (e.g., testing a series of oligonucleotides designed to be complementary to different regions of the targeted mRNA sequence). See Nielsen (1999) Current Opinion in Structural Bio. 9:353-357; Monia et al. (1996) Nat. Med. 2:668-675. It should be noted that the observation that in vitro PNA/mRNA hybrids are not a substrate for RNase H does not exclude the possibility that PNA binding in vivo could mediate degradation of the targeted mRNA by an alternative catalytic mechanism of action. However, it is likely that the efficiency of the antisense activity of a PNA antisense agent may rely on a mechanism that is related to the stability of the resulting PNA/mRNA hybrid.

PNA molecules are characterized by extremely desirable nucleic acid hybridization properties (e.g., high affinity and specificity) enabling them to form extremely stable duplex hybrids with complementary DNA, RNA or PNA oligomer sequences. In fact, the sequence discrimination (i.e., specificity) of PNA/DNA binding has been systematically determined to be as high or even higher than that of DNA (Larsen et al. (1999) Biochem. Et Biophysica Acta 1489:159-166). In addition, the peptide (or amide) bonds in PNAs are sufficiently distinct from the alpha-amino acid peptide bonds present in protein to confer protease- and peptidase-resistance to peptide nucleic acid molecules. Thus, PNA oligomers are highly stable in biological environments.

These inherent characteristics (e.g., high affinity, specificity, and biological stability) make PNA molecules attractive alternative agent for use as an antisense agent for the sequence-specific (i.e., based on specific hybridization) regulation of a target mRNA and its encoded protein. However, unlike other nucleic acid analogs, PNA molecules are not spontaneously taken up by all cell types. This limitation can be obviated by the use of a cell-penetrating transit peptide (e.g., transportan or antennapedia (pAntp). See, for example, Pooga et al. (1998) Nature Biotechnology 16:877. It has recently been demonstrated that PNA-peptide conjugates are efficiently taken up by certain eukaryotic cells in vitro (Aldrian-Herrada et al. (1998) Nucleic Acids Res. 26(21): 4910) and that such agents can be employed to mediate the down regulation of target genes in nerve cell cultures. (Nielsen (1999) Current Opin. Structural Bio. 9:353-357).

Investigators have also recently reported in vivo biological activity of antisense PNA and PNA-peptide conjugates targeted to neuronal receptors (Pooga et al. (1998) Natl. Biotechnol 16:857; Tyler et al. (1998) FEBS Lett 421:280-284). More specifically, Pooga et al. report that a PNA antisense oligomer specific for the galanin receptor coupled to the cell-penetrating peptide antennapedia, delivered by intrathecal injection, inhibited galatin receptor expression in vivo in rat spinal cords, and demonstrated that the reduced receptor levels contributed to a modified pain response. Tyler et al. report that “naked PNA” (e.g., PNA molecules that are not conjugated to a transit peptide) are taken up by neuronal cells in vivo (Tyler et al. (1998) FEBS Lett. 421:280-284). More specifically, Tyler et al utilized a short (e.g., 12-14 mer) PNA molecule to target the neurotensin receptor (NTR-1) and the mu opiod receptor in the brain of rats. Therefore, it may be possible to utilize the delivery method of the invention to deliver antisense PNA molecules directly to the mammalian CNS and to effectively inhibit protein expression therein. Considered together, these data demonstrate that PNAs readily enter neuronal cells in vivo and suggest that antisense PNA agents delivered to the CNS may function as an effective and specific regulatory agent.

The synthesis of polynucleotide mimics contemplated for use in the method of the present invention can be performed either with Boc-, Fmoc-, or -protected monomers according to conventional solid-phase peptide technologies and are purified by reversed-phase high-performance liquid chromatography (RP-HPLC) using techniques that are well known to one of skill in the art. In addition, because PNA oligomers are synthesized by conventional peptide chemistry protocols, it is relatively easy to conjugate a peptide to a particular PNA oligomer thereby producing a PNA-peptide conjugate. For example, a peptide embodying a carrier moiety could be conjugated to a PNA oligomer to facilitate cellular uptake or membrane transport of the oligomer. Alternatively, PNA monomers and/or oligomers designed for regulation of a target RNA can be prepared by a commercial supplier.

Administering the Polynucleotide Agent

For a therapeutic embodiment of the invention, the total amount of polynucleotide agent administered per dose should be in a range sufficient to deliver a biologically relevant amount of the agent. For example, the total amount of agent administered per dose could range from about 1 μM to about 100 μM (e.g., about 1 μM, 5 μM, 10 μM, 20 μM, 25 μM, 30 μM, 40 μM, 50 μM, 65 μM, 75 μM, 80 μM, 90 μM or 100 μM). The pharmaceutical composition having a unit dose of agent can be in the form of a solution, suspension, emulsion, powder, microparticle, or a sustained-release formulation. The total volume of the pharmaceutical composition administered can range from about 10 l to about 1000 pi. For example, a single dose of an aqueous solution administered to the olfactory region of the nasal cavity, can range from about 10 μl to about 200 μl. It is apparent that the suitable volume can vary with factors such as the size of the tissue to which the agent is administered and the solubility of the agents in the composition. Nasal administration may require the administration of more than one dose, for example two or more doses may be administered.

It is recognized that the total amount of agent administered as a unit dose to a particular tissue will depend upon the type of pharmaceutical composition being administered, that is whether the composition is in the form of, for example, a solution, a suspension, an emulsion, a powder, a microparticle, or a sustained-release formulation. Needle-free subcutaneous administration to an extranasal tissue innervated by the trigeminal nerve may be accomplished by use of a device that employs a supersonic gas jet as a power source to accelerate an agent that is formulated as a powder or a microparticle into the skin. The characteristics of such a delivery method will be determined by the properties of the particle, the formulation of the agent, and the gas dynamics of the delivery device. Similarly, the subcutaneous delivery of an aqueous composition can be accomplished in a needle-free manner by employing a gas-spring powered hand-held device to produce a high force jet of fluid capable of penetrating the skin. Alternatively, a skin patch formulated to mediate a sustained release of a composition can be employed for the transdermal delivery of an agent to a tissue innervated by the trigeminal nerve. Where the pharmaceutical composition comprises a therapeutically effective amount of an agent, or a combination of agents, in a sustained-release formulation, the agent(s) is/are administered at a higher concentration.

It should be apparent to a person skilled in the art that in order to obtain continuous suppression of the target gene chronic or repeated delivery of an antisense agent may be required, due to the transient nature of gene expansion. For example, antisense inhibition of a gene product with a long half-life, for example a membrane receptor, could require several administrations where as the amount of agent required to inhibit production of a protein with a rapid turnover, may require only a single administration or a cyclic administration. Accordingly, variations may be acceptable with respect to the therapeutically effective dose and frequency of the administration of an antisense agent in this embodiment of the invention. The amount of the agent administered will be inversely correlated with the frequency of administration. Hence, an increase in the concentration of agent in a single administered dose, or an increase in the mean residence time in the case of a sustained-release form of agent, generally will be coupled with a decrease in the frequency of administration.

In the practice of the present invention, additional factors should be taken into consideration when determining the therapeutically effective dose of agent and frequency of its administration. Such factors include, for example, the size of the tissue, the area of the surface of the tissue, the severity of the disease or disorder, and the age, height, weight, health, and physical condition of the individual to be treated. Generally, a higher dosage is preferred if the tissue is larger or the disease or disorder is more severe.

Some minor degree of experimentation may be required to determine the most effective dose and frequency of dose administration, this being well within the capability of one skilled in the art once apprised of the present disclosure.

Pharmaceutical Composition

The delivery method of the present invention can be employed to administer an effective amount of a pharmaceutical composition comprising a polynucleotide agent to the CNS. The invention is, in particular, directed to a method that can be employed for the direct delivery of compositions comprising a polynucleotide agent that either codes for a protein or a peptide or is designed to be complementary to the sequence of an endogenous mRNA sequence to the CNS, brain, and/or spinal cord. As used herein the terms “effective amount” and “therapeutically effective dose” refer to achieving a level (concentration of peptide or protein or level of inhibition of protein expression) sufficient to prevent, treat, reduce, and/or ameliorate the symptoms and/or underlying causes of any of the disorders or diseases described elsewhere herein. In some instances, an “effective amount” is sufficient to eliminate the symptoms of those diseases and, perhaps, overcome the disease itself. In the context of the present invention, the terms “treat” and “therapy” and the like refer to alleviate, slow the progression, prophylaxis, attenuation, or cure of existing disease. Prevent, as used herein, refers to putting off, delaying, slowing, inhibiting, or otherwise stopping, reducing, or ameliorating the onset of such CNS diseases or disorders. It is preferred that a large enough quantity of the agent be applied in non-toxic levels in order to provide an effective level of activity within the neural system against the disease. The method of the present invention may be used with any mammal. Exemplary mammals include, but are not limited to rats, cats, dogs, horses, cows, sheep, pigs, and more preferably humans.

For polynucleotide agents administered by way of an intranasal route, it is preferred that the agent be capable of at least partially dissolving in the fluids that are secreted by the mucous membrane that surrounds the cilia of the olfactory receptor cells of the neuroepithelium. The composition can include, for example, any pharmaceutically acceptable additive, carrier, or adjuvant that facilitates the agent's dissolution or transport and which is suitable for administration to a tissue innervated by the olfactory and/or trigeminal nerves. Preferably, the pharmaceutical composition can be employed for the prevention or treatment of a disorder, malignancy (e.g., a solid tumor), disease or injury of the CNS, brain, and/or spinal cord. Preferably, the composition includes a agent in combination with a pharmaceutical carrier, additive, and/or adjuvant that can promote the transfer of the agent within or through tissue innervated by the olfactory and/or trigeminal nerves. Alternatively, the agent may be combined with substances that may assist in transporting the agent to sites of nerve cell damage. The composition can include one or several antisense agents.

The composition typically contains a pharmaceutically acceptable carrier mixed with the polynucleotide agent and other components in the pharmaceutical composition. By “pharmaceutically acceptable carrier” is intended a carrier that is conventionally used in the art to facilitate the storage, administration, and/or the healing effect of the agent. A carrier may also reduce any undesirable side effects of the agent. A suitable carrier should be stable, i.e., incapable of reacting with other ingredients in the formulation. It should not produce significant local or systemic adverse effect in recipients at the dosages and concentrations employed for treatment. Such carriers are generally known in the art. For example, a suitable carriers for this invention include those conventionally used for large stable macromolecules such as albumin, gelatin, collagen, polysaccharide, monosaccharides, polyvinylpyrrolidone, polylactic acid, polyglycolic acid, polymeric amino acids, fixed oils, ethyl oleate, liposomes, glucose, sucrose, lactose, mannose, dextrose, dextran, cellulose, mannitol, sorbitol, polyethylene glycol (PEG), and the like.

Water, saline, aqueous dextrose, and glycols are preferred liquid carriers, particularly (when isotonic) for solutions. The carrier can be selected from various oils, including those of petroleum, animal, vegetable or synthetic origin, for example, peanut oil, soybean oil, mineral oil, sesame oil, and the like. Suitable pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol, and the like. The compositions can be subjected to conventional pharmaceutical expedients, such as sterilization, and can contain conventional pharmaceutical additives, such as preservatives, stabilizing agents, wetting, or emulsifying agents, salts for adjusting osmotic pressure, buffers, and the like.

A composition formulated for intranasal delivery may optionally comprise an odorant. An odorant agent is combined with the neurologic agent to provide an odorliferous sensation, and/or to encourage inhalation of the intranasal preparation to enhance delivery of the active neurologic agent to the olfactory neuroepithelium. The odorliferous sensation provided by the odorant agent may be pleasant, obnoxious, or otherwise malodorous. The odorant receptor neurons are localized to the olfactory epithelium, which, in humans, occupies only a few square centimeters in the upper part of the nasal cavity. The cilia of the olfactory neuronal dendrites, which contain the receptors, are fairly long (about 30-200 um). A 10-30 um layer of mucus envelops the cilia, which the odorant agent must penetrate to reach the receptors. See Snyder et al. (1988) J Biol. Chem. 263:13972-13974. Use of a lipophillic odorant agent having moderate to high affinity for odorant binding protein (OBP) is preferred. OBP has an affinity for small lipophillic molecules found in nasal secretions and may act as a carrier to enhance the transport of a lipophillic odorant substance and active neurologic agent to the olfactory receptor neurons. It is also preferred that an odorant agent is capable of associating with lipophillic additives such as liposomes and micelles within the preparation to further enhance delivery of the neurologic agent by means of OBP to the olfactory neuroepithelium. OBP may also bind directly to lipophillic agents to enhance transport of the neurologic agent to olfactory neural receptors.

Suitable odorants having a high affinity for OBP include terpanoids such as cetralva and citronellol, aldehydes such as amyl cinnamaldehyde and hexyl cinnamaldehyde, esters such as octyl isovalerate, jasmines such as C1S-jasmine and jasmal, and musk 89. Other suitable odorant agents include those which may be capable of stimulating odorant-sensitive enzymes such as aderrylate cyslase and guanylate cyclase, or which may be capable of modifying ion channels within the olfactory system to enhance absorption of the neurologic agent.

Other acceptable components in the composition include, but are not limited to, pharmaceutically acceptable agents that modify isotonicity, including water, salts, sugars, polyols, amino acids, and buffers. Examples of suitable buffers include phosphate, citrate, succinate, acetic acid, and other organic acids or their salts. Typically, the pharmaceutically acceptable carrier also includes one or more stabilizers, reducing agents, anti-oxidants and/or anti-oxidant chelating agents. The use of buffers, stabilizers, reducing agents, anti-oxidants and chelating agents in the preparation of protein based compositions, particularly pharmaceutical compositions, is well known in the art. See, Wang et al. (1980) J. Parent. Drug Assn. 34(6):452-462 (1980); Wang et al. (1988) J. Parent. Sci. and Tech. 42:S4-S26; Lachman et al. (1968) Drug and Cosmetic Industry 102(1):36-38, 40 and 146-148; and Akers (1988) J. Parent. Sci. and Tech. 36(5):222-228.

Suitable buffers include acetate, adipate, benzoate, citrate, lactate, maleate, phosphate, tartarate, borate, tri(hydroxymethyl aminomethane), succinate, glycine, histidine, the salts of various amino acids, or the like, or combinations thereof. See Wang (1980) Supra, p. 455. Suitable salts and isotonicifiers include sodium chloride, dextrose, mannitol, sucrose, trehalose, or the like. Where the carrier is a liquid, it is preferred that the carrier is hypotonic or isotonic with oral, conjunctival, or dermal fluids and have a pH within the range of 4.5-8.5. Where the carrier is in powdered form, it is preferred that the carrier is also within an acceptable non-toxic pH range.

Suitable reducing agents, which maintain the reduction of reduced cysteines, include dithiothreitol (DTT also known as Cleland's reagent) or dithioerythritol at 0.01% to 0.1% wt/wt; acetylcysteine or cysteine at 0.1% to 0.5% (pH 2-3); and thioglycerol at 0.1% to 0.5% (pH 3.5 to 7.0) and glutathione. See Akers (1988) supra, pp. 225-226. Suitable antioxidants include sodium bisulfite, sodium sulfite, sodium metabisulfite, sodium thiosulfate, sodium formaldehyde sulfoxylate, and ascorbic acid. See Akers (1988) supra, p. 225. Suitable chelating agents, which chelate trace metals to prevent the trace metal catalyzed oxidation of reduced cysteines, include citrate, tartarate, ethylenediaminetetraacetic acid (EDTA) in its disodium, tetrasodium, and calcium disodium salts, and diethylenetriamine pentaacetic acid (DTPA). See, e.g., Wang (1980) supra, pp. 457-458 and 460-461, and Akers (1988) supra, pp. 224-227.

The composition can include one or more preservatives such as phenol, cresol, p-aminobenzoic acid, BDSA, sorbitrate, chlorhexidine, benzalkonium chloride, or the like. Suitable stabilizers include carbohydrates such as trehalose or glycerol. The composition can include a stabilizer such as one or more of microcrystalline cellulose, magnesium stearate, mannitol, sucrose to stabilize, for example, the physical form of the composition; and one or more of glycine, arginine, hydrolyzed collagen, or protease inhibitors to stabilize, for example, the chemical structure of the composition. Suitable suspending additives include carboxymethyl cellulose, hydroxypropyl methylcellulose, hyaluronic acid, alginate, chondroitin sulfate, dextran, maltodextrin, dextran sulfate, or the like. The composition can include an emulsifier such as polysorbate 20, polysorbate 80, pluronic, triolein, soybean oil, lecithins, squalene and squalanes, sorbitan treioleate, or the like. The composition can include an antimicrobial such as phenylethyl alcohol, phenol, cresol, benzalkonium chloride, phenoxyethanol, chlorhexidine, thinerosol, or the like. Suitable thickeners include natural polysaccharides such as mannans, arabinans, alginate, hyaluronic acid, dextrose, or the like; and synthetic ones like the PEG hydrogels of low molecular weight and aforementioned suspending agents.

The composition can include an adjuvant such as cetyl trimethyl ammonium bromide, BDSA, cholate, deoxycholate, polysorbate 20 and 80, fuisidic acid, or the like, and in the case of DNA delivery, preferably, a cationic lipid. Suitable sugars include glycerol, threose, glucose, galactose, mannitol, and sorbitol. A suitable protein is human serum albumin.

Preferred compositions include one or more of a solubility enhancing additive, preferably a cyclodextrin; a hydrophilic additive, preferably a mono succhamide or oligosaccharide; an absorption promoting additive, preferably a cholate, a deoxycholate, a fusidic acid, or a chitosan; a cationic surfactant, preferably a cetyl trimethyl ammonium bromide; a viscosity enhancing additive, preferably to promote residence time of the composition at the site of administration, preferably a carboxymethyl cellulose, a maltodextrin, an alginic acid, a hyaluronic acid, or a chondroitin sulfate; or a sustained release matrix, preferably a polyanhydride, a polyorthoester, a hydrogel, a particulate slow release depo system, preferably a polylactide co-glycolides (PLG), a depo foam, a starch microsphere, or a cellulose derived buccal system; a lipid-based carrier, preferably an emulsion, a liposome, a niosomes, or a micelles. The composition can include a bilayer destabilizing additive, preferably a phosphatidyl ethanolamine; a flsogenic additive, preferably a cholesterol hemisuccinate.

Other preferred compositions for sublingual administration include, for example, a bioadhesive to retain the agent sublingually; a spray, paint, or swab applied to the tongue; retaining a. slow dissolving pill or lozenge under the tongue; or the like. Other preferred compositions for transdermal administration include a bioadhesive to retain the agent on or in the skin; a spray, paint, cosmetic, or swab applied to the skin; or the like.

These lists of carriers and additives is by no means complete and a worker skilled in the art can choose excipients from the GRAS (generally regarded as safe) list of chemicals allowed in the pharmaceutical preparations and those that are currently allowed in topical and parenteral formulations.

For the purposes of this invention, the pharmaceutical composition including agent can be formulated in a unit dosage and in a form such as a solution, suspension, or emulsion. The agent may be administered to tissue innervated by the trigeminal and/or olfactory neurons as a powder, a granule, a solution, a cream, a spray (e.g., an aerosol), a gel, an ointment, an infusion, an injection, a drop, or sustained release composition, such as a polymer disk. For buccal administration, the compositions can take the form of tablets or lozenges formulated in a conventional manner. For administration to the eye or other external tissues, e.g., mouth and skin, the compositions can be applied to the infected part of the body of the patient as a topical ointment or cream. The compounds can be presented in an ointment, for instance with a water-soluble ointment base, or in a cream, for instance with an oil-in-water cream base. For conjunctival applications, the agent can be administered in biodegradable or non-degradable ocular inserts. The drug may be released by matrix erosion or passively through a pore as in ethylene-vinylacetate polymer inserts. For other mucosal administrations, such as sublingual, powder discs may be placed under the tongue and active delivery systems may for in situ by slow hydration as in the formulation of liposomes from dried lipid mixtures or pro-liposomes.

Other preferred forms of compositions for administration include a suspension of a particulate, such as an emulsion, a liposome, an insert that releases the agent slowly, and the like. The powder or granular forms of the pharmaceutical composition may be combined with a solution and with a diluting, dispersing, or surface-active agent. Additional preferred compositions for administration include a bioadhesive to retain the agent at the site of administration; a spray, paint, or swab applied to the mucosa or epithelium; a slow dissolving pill or lozenge; or the like. The composition can also be in the form of lyophilized powder, which can be converted into a solution, suspension, or emulsion before administration. The pharmaceutical composition including agent is preferably sterilized by membrane filtration and is stored in unit-dose or multi-dose containers such as sealed vials or ampoules.

The method for formulating a pharmaceutical composition is generally known in the art. A thorough discussion of formulation and selection of pharmaceutically acceptable carriers, stabilizers, and isomolytes can be found in Remington 's Pharmaceutical Sciences (18^{th} ed.; Mack Publishing Company, Eaton, Pa., 1990), herein incorporated by reference.

The polynucleotide agents of the present invention can also be formulated in a sustained-release form to prolong the presence of the pharmaceutically active agent in the treated mammal, generally for longer than one day. Many methods of preparation of a sustained-release formulation are known in the art and are disclosed in Remington 's Pharmaceutical Sciences (18^{th} ed.; Mack Publishing Company, Eaton, Pa., 1990), herein incorporated by reference.

Generally, the agent can be entrapped in semipermeable matrices of solid hydrophobic polymers. The matrices can be shaped into films or microcapsules. Examples of such matrices include, but are not limited to, polyesters, copolymers of L-glutamic acid and gamma ethyl-L-glutamate (Sidman et al. (1983) Biopolymers 22:547-556), polylactides (U.S. Pat. No. 3,773,919 and EP 58,481), polylactate polyglycolate (PLGA) such as polylactide-co-glycolide (see, for example, U.S. Pat. Nos. 4,767,628 and 5,654,008), hydrogels (see, for example, Langer et al. (1981) J. Biomed. Mater. Res. 15:167-277; Langer (1982) Chem. Tech. 12:98-105), non-degradable ethylene-vinyl acetate (e.g., ethylene vinyl acetate disks and poly(ethylene-co-vinyl acetate)), degradable lactic acid-glycolic acid copolyers such as the Lupron Depot™, poly-D-(−)-3-hydroxybutyric acid (EP 133,988), hyaluronic acid gels (see, for example, U.S. Pat. No. 4,636,524), alginic acid suspensions, and the like.

Suitable microcapsules can also include hydroxymethylcellulose or gelatin-microcapsules and polymethyl methacrylate microcapsules prepared by coacervation techniques or by interfacial polymerization. See International Publication Number WO 99/24061, “Method for Producing Sustained-release Formulations,” wherein a protein is encapsulated in PLGA microspheres, herein incorporated by reference. In addition, microemulsions or colloidal drug delivery systems such as liposomes and albumin microspheres, may also be used. See Remington's Pharmaceutical Sciences (18^{th} ed.; Mack Publishing Company Co., Eaton, Pa., 1990). Other preferred sustained-release compositions employ a bioadhesive to retain the agent at the site of administration.

Among the optional substances that may be combined with the agent in the pharmaceutical composition are lipophilic substances that can enhance absorption of the agent through the mucosa or epithelium of the nasal cavity, or along a neural, lymphatic, or perivascular pathway to damaged nerve cells in the CNS. The agent may be mixed with a lipophilic adjuvant alone or in combination with a carrier, or may be combined with one or several types of micelle or liposome substances. Among the preferred lipophilic substances are cationic liposomes including one or more of the following: phosphatidyl choline, lipofectin, DOTAP, a lipid-peptoid conjugate, a synthetic phospholipid such as phosphatidyl lysine, or the like. These liposomes may include other lipophilic substances such as gangliosides and phosphatidylserine (PS). Also preferred are micellar additives such as GM-1 gangliosides and phosphatidylserine (PS), which may be combined with the agent either alone or in combination. GM-I ganglioside can be included at 1-10 mole percent in any liposomal compositions or in higher amounts in micellar structures. Protein agents can be either encapsulated in particulate structures or incorporated as part of the hydrophobic portion of the structure depending on the hydrophobicity of the active agent. A preferred liposomal formulation employs Depofoam.

Intermittent Dosing

In another embodiment of the invention, the pharmaceutical composition comprising the therapeutically effective dose of agent is administered intermittently. By “intermittent administration” is intended administration of a therapeutically effective dose of agent, followed by a time period of discontinuance, which is then followed by another administration of a therapeutically effective dose, and so forth. Administration of the therapeutically effective dose may be achieved in a continuous manner, as for example with a sustained-release formulation, or it may be achieved according to a desired daily dosage regimen, as for example with one, two, three or more administrations per day. By “time period of discontinuance” is intended a discontinuing of the continuous sustained-released or daily administration of agent. The time period of discontinuance may be longer or shorter than the period of continuous sustained-release or daily administration. During the time period of discontinuance, the agent level in the relevant tissue is substantially below the maximum level obtained during the treatment. The preferred length of the discontinuance period depends on the concentration of the effective dose and the form of agent used. The discontinuance period can be at least 1 day, preferably is at least 2 day, more preferably is at least and generally does not exceed a time period of 1 week. When a sustained-release formulation is used, the discontinuance period must be extended to account for the greater residence time of agent at the site of injury. Alternatively, the frequency of administration of the effective dose of the sustained-release formulation can be decreased accordingly. An intermittent schedule of administration of agent can continue until the desired therapeutic effect, and ultimately treatment of the disease or disorder, is achieved.

In yet another embodiment, intermittent administration of the therapeutically effective dose of agent is cyclic. By “cyclic” is intended intermittent administration accompanied by breaks in the administration, with cycles ranging from about 2 to about 10. For example, the administration schedule might be intermittent administration of the effective dose of agent, wherein a single short-term dose is given once every 2 days, followed by a break in intermittent administration for a period of 1 week, followed by intermittent administration by administration of a single short-term dose given once per day for two weeks, followed by a break in intermittent administration for a period of two weeks, and so forth

The present invention may be better understood with reference to the following examples. These examples are intended to be representative of specific embodiments of the invention, and are not intended as limiting the scope of the invention.

### Experimental

Although the examples presented herein are limited to the delivery/administration of chimeric oligonucleotides, the invention should not be construed as being limited to this single class of polynucleotide agents.

Introduction

Intranasal administration is an effective means for delivering an antisense polynuceotide agent complementary to the IGF-1 receptor to the CNS. For a detailed description of the structure of the chimeric antisense oligonucleotides used in the examples below, see International Publication No. WO 01/16306 A2; and U.S. application Ser. Nos. 60/151,246, filed Aug. 27, 1999, and 09/648,254, filed Aug. 25, 2000, both entitled “Chimeric Antisense Oligonucleotides and Cell Transfecting Formulations Thereof,” herein incorporated by reference.

### EXAMPLE 1

### Delivery of 35S Antisense Oligonucleotide (AON) for the lGF-1 Receptor to the CNS by Intranasal Administration

Chimeric Antisense Oligonucleotides

In general, a chimeric antisense oligonucleotide suitable for use with the methods of the invention will have the structure shown below:
5′—W—X¹—Y—X²-Z-3′.
   In this structure, the central or core region of the molecule, represented by Y, is a block of about five to twelve phosphorothioate-linked deoxyribonucletides. Such sequences are known to activate RNAse H when hybridized to a complementary, or near-complementary strand of RNA, thus promoting cleavage of the target RNA. This region is flanked by two blocks, represented by X¹ and X², each having about seven to twelve phosphodiester-linked 2′-O-methyl ribonucleotide subunits. These regions, while not effective to activate RNAase H, provide high affinity binding to complementary or near complementary RNA strands and are generally characterized by reduced cellular toxicity compared to phosphorothioate-linked subunits.

The presence of the 2′-O-methyl substituents of the RNA subunits provides a moderate increase in stability, in comparison to the stability of unsubstituted (e.g., 2-hydroxy) ribose moieties; however, the phosphodiester 2′-O-methyl RNA subunits are nonetheless susceptible to attack by cellular exonucleases. Accordingly, a chimeric antisense oligonucleotide (AON) may optionally comprise blocking groups, designated as W and Z in the above representation, respectively, at the 5′ and 3′ termini. The blocking groups may be linked to their respective X blocks by phosphodiester linkages. The 3′-blocking group, Z, is preferably a 3′-to-3′ linked nucleotide, although one of skill in the art will readily recognize that this terminus may also be blocked with other groups. The 5′ terminus is blocked with a 5′-O-alkyl thymidine subunit, preferably a 5′-O-methyl thymidine.

³⁵S-AON

The ³⁵S-labelled antisense oligonucleotide (³⁵S-AON) used was the Na⁺ salt form of an oligonucleotide comprising a sequence that corresponds to SEQ ID NO.: 1. More specifically, the ³⁵S-AON had the following structure:

5′: 2′OMe [U*(ps) C*(ps) U*(ps) U(po) C(po) C(po)
U(po) C] (ps) A(ps) C(ps) A(ps) G(ps) A(ps) C(ps)
C(ps) T(ps) T(ps) 2′OMe[C(po) G(po) G(po) C(ps)
A(ps) A(ps)G] 3′

wherein * indicates the location of the ³⁵S label and (ps) and (po) designate phosphorothioate and phosphodiester linkages, respectively. The central portion (e.g., core region) of the molecule, represented by the region Y in the generalized schematic shown above contains nine phosphorothionate-linked nucleotides, which are represented by the bolded nucleotides in the above representation. The core region corresponds to nucleotides 9 to 17 of SEQ ID NO: 1. The 5′ flanking region of phosphodiester-linked 2′-O-methyl ribonucleotides corresponding to region XI in the above representation corresponds to nucleotides 1 through 8 of SEQ ID NO: 1. The 3′ flanking region of phosphodiester-linked 2′-O-methyl ribonucleotides corresponding to region X2 in the above representation corresponds to nucleotides 18 through 25 of SEQ ID NO: 1. The specific IGF-I receptor sequence targeted by the antisense oligonucleotide shown in SEQ ID NO: 1 corresponds to nucleotides 1025-1049 of the human insulin-like growth factor I receptor (GenBank Accession No. X04434 M24599/Locus HSIGFIRR). Following identification of the specific IGF-I receptor target sequence, oligonucleotide sequence information was provided to TriLink Biotechnologies, Inc., for preparation as a radiolabeled antisense agent. The ³⁵S-AON was prepared by TriLink Biotechologies Inc., using solid phase synthesis, according to established methodologies well known to one of skill in the art. The use of a radioactively tagged agent is the preferred molecule for in vivo pharmacokinetic research because it is accepted as the least intrusive means of adding a tracer to a molecule. A key consideration for the use of a radioactively labeled oligonucleotide for in vivo studies is to ensure that the radiolabel is non-exchangeable. TriLinks addresses this concern by incorporating the radiolabel into the oligomer during its synthesis.
Intranasal Delivery to the CNS

Male Sprague-Dawley rats weighing 162 g (rat #3), 321 g (rat #8) and 336 g (rat #2) were anesthetized with intraperitoneal sodium pentobarbital (50 mg/kg). AON delivery to the CNS was assessed after intranasal administration of a composition comprising ³⁵S-AON in combination with unlabeled AON in phosphate-buffered saline, pH 7.4. Rats were placed on their backs and administered ˜100 microliters of ³⁵S-AON to each naris over a period of 20-30 minutes, alternating drops every 2-3 minutes between the left and right nares. During the intranasal administration of this agent, one side of the nose and mouth were held closed. This method of administering the agent allows for both pressure and gravity to deliver the agent into the upper one third of the nasal cavity. Rats subsequently underwent perfusion-fixation within minutes following the completion of ³⁵S-AON administration. Perfusion-fixation was performed with 50-100 ml physiologic saline followed by 500 ml of fixative containing 1.25% glutaraldehyde and 1% paraformaldehyde in 0.1 M Sorenson's phosphate buffer, pH 7.4, prior to spinal cord dissection, and ³⁵S measurements were determined. Areas dissected included the spinal cord, olfactory bulbs, frontal cortex, anterior olfactory nucleus, hippocampal formation, choroid plexus, diencephalon, medulla, pons, and cerebellum.

Data for the intranasal (I.N.) delivery of an antisense oligonucleotide
(³⁵S-AON) for the IGF-1 receptor to the CNS.
Rat AON #3 (³⁵S-AON + rhAON) weight = 162.2 grams
133.45 nmoles delivered
Anesthetic: Sodium pentobarbitol (Nembutal) administered I.P. (50 mg/kg)
I.N. administration time = 30 minutes

| | Activity of Antisense 0.8690 dpm/fmole | | | | |
|---|---|---|---|---|---|
| Tissue Type | Weight | DPM | fmoles | pM | nM |
| Blood Sample | 0.22424 | 582.0 | 669.7 | 2986.4 | 3.0 |
| #1 (5:00) | | | | | |
| Blood Sample | 0.23434 | 434.8 | 500.3 | 2135.0 | 2.1 |
| #2 (11:00) | | | | | |
| Blood Sample | 0.23354 | 617.4 | 710.5 | 3042.3 | 3.0 |
| #3 (15:10) | | | | | |
| Blood Sample | 0.23866 | 1272.8 | 1464.6 | 6136.8 | 6.1 |
| #4 (20:20) | | | | | |
| Blood Sample | 0.22726 | 1386.4 | 1595.4 | 7020.1 | 7.0 |
| #5 (25:00) | | | | | |
| Left Olfactory | 0.03992 | 1995.7 | 2296.5 | 57528.2 | 57.5 |
| Bulb | | | | | |
| Right Olfactory | 0.04184 | 2076.4 | 2389.4 | 57107.2 | 57.1 |
| Bulb | | | | | |
| Frontal Cortex | 0.05090 | 5219.0 | 6005.8 | 117991.2 | 118.0 |
| Caudate/ | 0.01181 | 34.5 | 39.7 | 3362.6 | 3.4 |
| Putamen | | | | | |
| Ant. Olf. | 0.01898 | 437.0 | 502.9 | 26495.1 | 26.5 |
| Nucleus | | | | | |
| L. | 0.04026 | 433.5 | 498.9 | 12391.8 | 12.4 |
| Hippocampal | | | | | |
| Form. | | | | | |
| Diencephalon | 0.24938 | 2049.2 | 2358.2 | 9456.1 | 9.5 |
| Midbrain | 0.09474 | 718.2 | 826.5 | 8723.7 | 8.7 |
| Pons | 0.05816 | 916.4 | 1054.6 | 18132.6 | 18.1 |
| Medulla | 0.14136 | 1895.9 | 2181.7 | 15433.8 | 15.4 |
| Cerebellum | 0.29641 | 1395.4 | 1605.7 | 5417.3 | 5.4 |
| Ventral Dura | 0.00204 | 949.4 | 1092.5 | 535532.2 | 535.5 |
| Trigeminal | 0.03227 | 6885.9 | 7923.9 | 245549.4 | 245.5 |
| Nerve | | | | | |
| Spinal Dura | 0.02060 | 45.0 | 51.8 | 2512.7 | 2.5 |
| Cervical Spinal | 0.15305 | 5851.9 | 6734.1 | 43999.2 | 44.0 |
| Cord | | | | | |
| Lumbar Spinal | 0.07121 | 0.0 | 0.0 | 0.0 | 0.0 |
| Cord | | | | | |
| Deltoid Muscle | 0.12213 | 43.4 | 50.0 | 409.0 | 0.4 |
| Liver | 0.13427 | 2873.2 | 3306.3 | 24624.2 | 24.6 |
| Kidney | 0.11940 | 2481.7 | 2855.8 | 23917.5 | 23.9 |
| Lung | 0.04468 | 31.7 | 36.5 | 817.0 | 0.8 |
| Esophagus | 0.02883 | 1844.5 | 2122.6 | 73623.5 | 73.6 |
| Trachea | 0.02847 | 179.6 | 206.7 | 7258.6 | 7.3 |
| R. Olfact. | 0.05196 | 978859.4 | 1126420.5 | 21678608.7 | 21678.6 |
| Epithelium | | | | | |

radioactivity administered = 52.24 uCi
72.38 dpm was subtracted as background from the original dpm.

Data for the intranasal (I.N.) delivery of an antisense oligonucleotide
(AON) for the IGF-1 receptor to the CNS.
Rat AON #2 (³⁵S-AON + rhAON) weight = 336.0 grams
68.218 nmoles delivered
Anesthetic: Sodium pentobarbitol (Nembutal) administered I.P. (50 mg/kg)
I.N. administration time = 13 minutes

| | Activity of Antisense 0.8526 dpm/fmole | | | | |
|---|---|---|---|---|---|
| Tissue Type | Weight | DPM | fmoles | pM | nM |
| Blood Sample | 0.22799 | 95.3 | 111.8 | 490.2 | 0.5 |
| #1 (5:00) | | | | | |
| Blood Sample | 0.23943 | 113.2 | 132.7 | 554.3 | 0.6 |
| #2 (10:00) | | | | | |
| Left Olfactory | 0.03992 | 825.0 | 967.6 | 24238.6 | 24.2 |
| Bulb | | | | | |
| Right Olfactory | 0.03336 | 826.6 | 969.5 | 29060.5 | 29.1 |
| Bulb | | | | | |
| Frontal Cortex | 0.02123 | 181.8 | 213.2 | 10044.4 | 10.0 |
| Caudate/Putamen | 0.00535 | 62.4 | 73.2 | 13684.4 | 13.7 |
| L. Hippocampal | 0.06674 | 972.5 | 1140.6 | 17090.1 | 17.1 |
| Form. | | | | | |
| R. Hippocampal | 0.08740 | 1226.1 | 1438.1 | 16454.0 | 16.5 |
| Form. | | | | | |
| Diencephalon | 0.32489 | 1844.6 | 2163.5 | 6659.1 | 6.7 |
| Midbrain | 0.05322 | 580.5 | 680.9 | 12793.5 | 12.8 |
| Pons | 0.06507 | 695.6 | 815.9 | 12538.1 | 12.5 |
| Medulla | 0.15719 | 2067.2 | 2424.6 | 15424.5 | 15.4 |
| Cerebellum | 0.24087 | 2400.9 | 2816.0 | 11690.8 | 11.7 |
| Ventral Dura | 0.00263 | 204.6 | 239.9 | 91226.2 | 91.2 |
| Trigeminal Nerve | 0.02787 | 332.5 | 390.0 | 13993.0 | 14.0 |
| Spinal Dura | 0.00564 | 46.0 | 53.9 | 9564.0 | 9.6 |
| Cervical Spinal | 0.08452 | 42.1 | 49.4 | 584.2 | 0.6 |
| Cord | | | | | |
| Thoracic Spinal | 0.08002 | 25.8 | 30.2 | 377.6 | 0.4 |
| Cord | | | | | |
| Lumbar Spinal | 0.09849 | 3.3 | 3.9 | 39.7 | 0.0 |
| Cord | | | | | |
| Deltoid Muscle | 0.07555 | 0.0 | 0.0 | 0.0 | 0.0 |
| Liver | 0.10652 | 17.7 | 20.7 | 194.3 | 0.2 |
| Kidney | 0.16888 | 31.6 | 37.0 | 219.1 | 0.2 |
| Lung | 0.11342 | 82.3 | 96.6 | 851.5 | 0.9 |
| Esophagus | 0.04848 | 72.6 | 85.1 | 1755.7 | 1.8 |
| Trachea | 0.04352 | 86.6 | 101.5 | 2333.4 | 2.3 |
| L. Olfact. | 0.06016 | 468011.0 | 548922.1 | 9124370.0 | 9124.4 |
| Epithelium | | | | | |
| R. Olfact. | 0.10629 | 495700.7 | 581398.9 | 5469930.1 | 5469.9 |
| Epithelium | | | | | |

Radioactivity: 1.0 uCi/ul 35S-AON (72.1 uCi)
70 dpm background was subtracted from the original dpm reading.

Data for the intranasal (I.N.) delivery of an antisense oligonucleotide
(AON) for the IGF-1 receptor to the CNS.
Rat AON #8 (³⁵S-AON + rhAON) weight = 321.9 grams
133.45 nmoles delivered
Anesthetic: Sodium pentobarbitol (Nembutal) administered I.P. (50 mg/kg)
I.N. administration time = 25 minutes

| | Activity of Antisense 0.7743 dpm/fmole | | | | |
|---|---|---|---|---|---|
| Tissue Type | Weight | DPM | Fmoles | pM | nM |
| Blood Sample #1 (5:50) | 0.24895 | 1904.7 | 2459.9 | 9881.1 | 9.9 |
| Blood Sample #2 (10:00) | 0.23735 | 3149.5 | 4067.7 | 17137.3 | 17.1 |
| Blood Sample #3 (15:00) | 0.25270 | 3172.1 | 4096.7 | 16211.8 | 16.2 |
| Blood Sample #4 (20:00) | 0.22984 | 2411.3 | 3114.2 | 13549.3 | 13.5 |
| Blood Sample #5 (26:00) | 0.23207 | 2300.9 | 2971.6 | 12804.7 | 12.8 |
| Left Olfactory Bulb | 0.03864 | 3648.2 | 4711.6 | 121936.1 | 121.9 |
| Right Olfactory Bulb | 0.02086 | 2483.3 | 3207.2 | 153746.6 | 153.7 |
| Frontal Cortex | 0.08305 | 818.5 | 1057.1 | 12728.3 | 12.7 |
| Caudate/Putamen | 0.01548 | 0.0 | 0.0 | 0.0 | 0.0 |
| Ant. Olf. Nucleus | 0.01758 | 21.7 | 28.0 | 1594.2 | 1.6 |
| Left Hippocampal Form. | 0.26831 | 225.9 | 291.7 | 1087.4 | 1.1 |
| Right Hippocampal Form | 0.09426 | 147.0 | 189.8 | 2014.1 | 2.0 |
| Diencephalon | 0.21560 | 626.6 | 809.2 | 3753.5 | 3.8 |
| Midbrain | 0.08258 | 256.3 | 331.0 | 4008.3 | 4.0 |
| Pons | 0.09447 | 221.3 | 285.8 | 3025.4 | 3.0 |
| Medulla | 0.12234 | 364.0 | 470.1 | 3842.6 | 3.8 |
| Cerebellum | 0.27143 | 1744.0 | 2262.4 | 8298.1 | 8.3 |
| Trigeminal Nerve | 0.03627 | 1246.9 | 1610.4 | 44399.2 | 44.4 |
| Spinal Dura | 0.03604 | 3.8 | 4.9 | 136.2 | 0.1 |
| Cervical Spinal Cord | 0.04376 | 67.2 | 86.8 | 1983.3 | 2.0 |
| Thoracic Spinal Cord | 0.05743 | 18.8 | 24.3 | 422.8 | 0.4 |
| Lumbar Spinal Cord | 0.04724 | 0.0 | 0.0 | 0.0 | 0.0 |
| Deltoid Muscle | 0.20364 | 71.1 | 91.8 | 450.9 | 0.5 |
| Liver | 0.13325 | 355.0 | 458.5 | 3440.7 | 3.4 |
| Kidney | 0.06962 | 163.3 | 210.9 | 3029.3 | 3.0 |
| Lung | 0.04366 | 25.8 | 33.3 | 763.2 | 0.8 |
| Esophagus | 0.03332 | 4895.2 | 6322.1 | 189738.8 | 189.7 |
| Trachea | 0.02673 | 18806.2 | 24288.0 | 908642.1 | 908.6 |
| L. Olfact. Epithelium | 0.04038 | 4051506.0 | 5232475.8 | 129580876.3 | 129580.9 |
| R. Olfact. Epithelium | 0.05933 | 86.0 | 111.1 | 1872.0 | 1.9 |

Radioactivity administered = 49.5 uCi (1.575 nmoles/ul; 84 ul total volume administered)
70 dpm background was subtracted from the original dpm.

### EXAMPLE 2

### Delivery of ³H AON for the IGF-1 Receptor to the CNS by Intranasal Administration

Preparation of ³H-AON

The ³H-labelled antisense oligonucleotide (³H-AON) used was the Na⁺ salt form of an oligonucleotide comprising a sequence which corresponds to SEQ ID NO: 1 and has the following structure:

5′: (5′-OMe-T) 2′Ome [UCUUCCUC]ps A(ps) C(ps)
A(ps) G(ps) A(ps) C(ps) C(ps) T*(ps) T(ps) 2′OMe
[CGGGCA] 3′-3′-G

wherein * indicates the location of the non-exchangeable tritium label and (ps) and (po) designate phosphorothioate and phosphodiester linkages, respectively. The core region of the molecule contains the same nine phosphorothionate-linked core nucleotides as the described above for the core region of the ³⁵S-AON. As stated above for the ³⁵S-AON, the core nucleotides, which are represented by the bolded nucleotides in the above representation, correspond to nucleotides 9 through 17 of SEQ ID NO:1; X¹ corresponds to nucleotides 1 through 8 of SEQ ID NO: 1; and X² corresponds to nucleotides 18 through 25 of SEQ ID NO: 1. The ³H-AON was prepared by TriLink Biotechnologies Inc. using solid phase synthesis, according to established methodologies well known to one of skill in the art.
Intranasal Delivery to the CNS:

A male Sprague-Dawley rat weighing 463.5 g (rat #1) was anesthetized with intraperitoneal sodium pentobarbital (50 mg/kg). AON delivery to the CNS was assessed after intranasal administration of 143 nmoles of a composition comprising ³H-AON in combination with unlabeled AON in phosphate-buffered saline, pH 7.4. The rat was placed on its back and administered 100 microliters of ³⁵S-AON to each naris over a period of 20-30 minutes, alternating drops every 2-3 minutes between the left and right nares. The rat subsequently underwent perfusion-fixation within minutes following the completion of ³H-AON administration. Perfusion-fixation was performed with 50-100 ml physiologic saline followed by 500 ml of fixative containing 1.25% glutaraldehyde and 1% paraformaldehyde in 0.1 M Sorenson's phosphate buffer, pH 7.4, prior to spinal cord dissection, and ³H measurements were determined. Areas dissected included the spinal cord, olfactory bulbs, frontal cortex, anterior olfactory nucleus, hippocampal formation, choroid plexus, diencephalon, medulla, pons, and cerebellum.

Data for the intranasal (I.N.) delivery of an antisense oligonucleotide
(AON) for the IGF-1 receptor to the CNS.
Rat AON #1 weight = 463.5 grams
143.75 nmoles administered (23.7 nmoles ³H-AON, 120 nmoles AON)
Anesthetic: Sodium pentobarbitol (Nembutal) administered I.P. (50 mg/kg)
I.N. administration time = 26 minutes

| | Activity of Antisense 0.2320 Dpm/fmole | | | | |
|---|---|---|---|---|---|
| Tissue Type | Weight | DPM | fmoles | pM | nM |
| Blood Sample | 0.22568 | 112.0 | 482.7 | 2138.9 | 2.1 |
| #1 (5:00) | | | | | |
| Blood Sample | 0.23388 | 27.8 | 119.7 | 511.8 | 0.5 |
| #2 (11:30) | | | | | |
| Blood Sample | 0.18051 | 47.6 | 205.1 | 1136.1 | 1.1 |
| #3 (17:30) | | | | | |
| Left Olfactory | 0.04066 | 255.0 | 1099.2 | 27033.5 | 27.0 |
| Bulb | | | | | |
| Right Olfactory | 0.04034 | 126.8 | 546.4 | 13544.4 | 13.5 |
| Bulb | | | | | |
| Frontal Cortex | 0.04195 | 667.7 | 2878.1 | 68606.9 | 68.6 |
| Caudate/ | 0.00621 | 45.4 | 195.6 | 31491.2 | 31.5 |
| Putamen | | | | | |
| Ant. Olf. | 0.00462 | 422.9 | 1822.8 | 394545.8 | 394.5 |
| Nucleus | | | | | |
| L. | 0.03265 | 158.2 | 682.1 | 20890.3 | 20.9 |
| Hippocampal | | | | | |
| Form. | | | | | |
| Diencephalon | 0.27263 | 714.5 | 3079.5 | 11295.6 | 11.3 |
| Midbrain | 0.04573 | 570.9 | 2460.9 | 53812.9 | 53.8 |
| Pons | 0.12760 | 573.8 | 2473.2 | 19382.7 | 19.4 |
| Medulla | 0.20795 | 866.3 | 3734.2 | 17957.3 | 18.0 |
| Cerebellum | 0.28751 | 1364.5 | 5881.3 | 20455.8 | 20.5 |
| Trigeminal | 0.01708 | 122.6 | 528.2 | 30927.0 | 30.9 |
| Nerve | | | | | |
| Spinal Dura | 0.02038 | 26.0 | 112.0 | 5494.7 | 5.5 |
| Cervical Spinal | 0.19925 | 130.8 | 563.6 | 2828.5 | 2.8 |
| Cord | | | | | |
| Thoracic Spinal | 0.08677 | 16.2 | 69.7 | 803.3 | 0.8 |
| Cord | | | | | |
| Lumbar Spinal | 0.55424 | 22.3 | 96.3 | 173.7 | 0.2 |
| Cord | | | | | |
| Deltoid Muscle | 0.18504 | 27.5 | 118.7 | 641.5 | 0.6 |
| Liver | 0.55424 | 33.9 | 145.9 | 263.3 | 0.3 |
| Kidney | 0.30004 | 33.3 | 143.7 | 478.8 | 0.5 |
| Lung | 0.09829 | 28.3 | 121.9 | 1240.6 | 1.2 |
| Esophagus | 0.04337 | 896.7 | 3865.1 | 89118.9 | 89.1 |
| Trachea | 0.04526 | 1134.6 | 4890.3 | 108049.1 | 108.0 |
| L. Olfact. | 0.05348 | 146338.9 | 630771.1 | 11794523.6 | 11794.5 |
| Epithelium | | | | | |
| R. Olfact. | 0.04189 | 76454.7 | 329545.9 | 7866935.0 | 7866.9 |
| Epithelium | | | | | |

Radioactivity: 0.5 uCi/ul
30 ul 3H-AON (15 uCi total) added to 60 ul (120 nmoles) AON
Total volume of mixture actually administered = 68 ul
19.28 dpm background subtracted from original dpm.

Conclusions

The data presented clearly demonstrate that antisense oligonucleotide is rapidly delivered to the brain and spinal cord within 30 minutes following intranasal administration. The rapid delivery to the olfactory bulb and anterior olfactory nucleus provides evidence for delivery along the olfactory neural pathway from the upper third of the nasal cavity to the brain. The rapid delivery to the trigeminal nerve, pons, midbrain, medulla, diencephalon, cerebellum and spinal cord provides evidence for delivery along the trigeminal neural pathway from the nasal cavity to the brain and spinal cord. Significant concentrations of antisense oligonucleotide are obtained not only in the above regions of the CNS, but also in the hippocampus and caudate/putamen.

Delivery is documented with both [³H] antisense oligonucleotide (AON#l) and [³⁵S] antisense oligonucleotide (AON#1). Delivery appears to be dose dependent as the average olfactory bulb concentration seen following delivery of 68.2 n moles was 27 nM (AON#1) as compared to 57 nM following delivery of 133 n moles (AON#1).

Demonstration of noninvasive delivery of antisense oligonucleotide to the CNS will improve the treatment and prevention of CNS disorders as it targets the CNS, reduces systematic side effects by reducing the amount of drug that enters the circulatory system, and allows for delivery of antisense agents that do not pass the BBB.

It should be noted that, as used in this specification the singular forms “a,” “an,” and “the” include plural referents unless the content clearly dictates otherwise. Thus, for example, reference to a composition containing “a compound” includes a mixture of two or more compounds.

All publications and patent applications in this specification are indicative of the level of ordinary skill in the art to which this invention pertains. All publications and patent applications are herein incorporated by reference in their entirety to the same extent as if each individual publication or patent application was specifically and individually indicated to be incorporated by reference in its entirety.

The invention has been described with reference to various specific and preferred embodiments and techniques. However, it should be understood that many variations and modifications may be made while remaining within the spirit and scope of the appended claims.

1-23. (canceled)

24. A method for delivering a polynucleotide agent to the central nervous system of a mammal, comprising: contacting an olfactory region of a nasal cavity or a tissue innervated by a trigeminal nerve with a composition comprising the agent whereby the agent is delivered to the tissues and cells of the central nervous system.

25. The method of claim 24, wherein the olfactory region comprises a nerve pathway, an epithelial pathway, a lymphatic channel, a perivascular channel, or a combination thereof.

26. The method of claim 24, wherein the composition comprising the polynucleotide agent is contacted with the mammal's olfactory region by administering the composition into an upper third of the nasal cavity.

27. The method of claim 24, wherein the tissue innervated by the trigeminal nerve is an intranasal tissue or an extranasal tissue selected from the group consisting of an oral tissue, a dermal tissue, or a conjunctiva.

28. The method of claim 24, wherein contacting the composition with the oral tissue comprises sublingual administration.

29. The method of claim 24, wherein the polynucleotide agent is delivered to a spinal cord, a brain stem, a mid-brain, a cerebellum, an olfactory bulb, a cortical structure, a subcortical structure, or any combination thereof.

30. The method of claim 24, wherein the polynucleotide agent is selected from the group consisting of a polynucleotide, a polynucleotide analogue, a polynucleotide mimic, and a plasmid operatively coding for a biologically active peptide or protein.

31. A method of inhibiting translation of a mRNA that encodes a target protein that contributes to the pathology of a central nervous system disorder of a mammal, comprising: providing a composition comprising at least one antisense agent that is complementary to a region of the mRNA; and contacting the composition with an olfactory region of the mammal's nasal cavity or with a tissue that is innervated by the trigeminal nerve, whereby the antisense agent is delivered to a cell of the central nervous system that comprises the mRNA, wherein the antisense agent hybridizes to the target mRNA and inhibits translation.

32. The method of claim 31, wherein the antisense agent is selected from the group consisting of an oligonucleotide, a chemically modified oligonucleotide, and a peptide nucleic acid molecule.

33. The method of claim 31, wherein the composition comprising the antisense molecule is contacted with the mammal's olfactory region by administering the composition into an upper third of the nasal cavity.

34. The method of claim 31, wherein the target protein is selected from the group consisting of: an insulin-like growth factor receptor I (IGF-IR), insulin-like growth factor-I (IGF-I), insulin-like growth factor II (IGF-II), an insulin-like growth factor-II (IGF-II) receptor, a Beta-Amyloid Precursor Protein, and an opiate receptor.

35. The method of claim 31, wherein the tissue innervated by the trigeminal nerve is an intranasal tissue or an extranasal tissue selected from the group consisting of an oral tissue, a dermal tissue, or a conjunctiva.

36. The method of claim 31, wherein contacting the composition with the oral tissue comprises sublingual administration.


SEQUENCE LISTING (9 sequences)

SEQ ID NO: 1 - DNA, 25 bp, Artificial Sequence
  Antisense sequence
tcttcctcacagaccttcgggcaag

SEQ ID NO: 2 - DNA, 18 bp, Artificial Sequence
  Antisense sequence
tcctccggagccagactt

SEQ ID NO: 3 - DNA, 18 bp, Artificial Sequence
  Antisense sequence
ggaccctcctccggagcc

SEQ ID NO: 4 - DNA, 17 bp, Artificial Sequence
  Antisense sequence
ccggagccagacttcat

SEQ ID NO: 5 - DNA, 20 bp, Artificial Sequence
  Antisense sequence
ctgctcctcctctaggatga

SEQ ID NO: 6 - DNA, 15 bp, Artificial Sequence
  Antisense sequence
ccctcctccggagcc

SEQ ID NO: 7 - DNA, 17 bp, Artificial Sequence
  Antisense sequence
tacttcagaccgaggcc

SEQ ID NO: 8 - DNA, 18 bp, Artificial Sequence
  Antisense sequence
ccgaggcctcctcccagg

SEQ ID NO: 9 - DNA, 18 bp, Artificial Sequence
  Antisense sequence
tcctccggagccagactt